(19) United States
(12) Patent Application Publication
(10) Pub. No.: US 2018/0231556 A1
(43) Pub. Date: Aug. 16, 2018

(54) METHODS OF DIAGNOSING AND TREATING CANCER

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR)

(72) Inventors: Marc POIROT, Toulouse (FR); Sandrine POIROT, Toulouse (US)

(21) Appl. No.: 15/946,789

(22) Filed: Apr. 6, 2018

Related U.S. Application Data
(63) Continuation of application No. 15/508,316, filed on Mar. 2, 2017.

(30) Foreign Application Priority Data
Sep. 5, 2014 (EP) 14306364.2

(51) Int. Cl. G01N 33/574; C12Q 1/6886

(52) U.S. Cl. CPC G01N 33/57415; C12Q 1/6886; C12Q 2600/158; G01N 2333/902; C12Q 2600/106

(57) ABSTRACT

The present invention relates to methods for the diagnosis and the treatment of cancer, in particular breast cancer. In particular, the present invention relates to a method of diagnosing cancer in a subject comprising the steps of i) determining the expression level of 11βHSD1 and/or 11βHSD2 in a sample obtained from the subject, ii) comparing the expression level determined at step i) with its predetermined reference value and ii) concluding that the subject suffers from a cancer when the expression level of 11βHSD1 is lower than its predetermined reference value or when the expression level of 11βHSD2 is higher than its predetermined reference value.

### FIELD OF THE INVENTION

The present invention relates to methods for the diagnosis and the treatment of cancer, in particular breast cancer.

### BACKGROUND OF THE INVENTION

Breast cancer (BC) is the most common female cancer. It affects more than 1 million women worldwide and about 400 000 subjects die due to this disease every year. Tamoxifen (Tam) is one of the major drugs used over the world for the therapy and prevention of breast cancers. In clinical practice, levels of estrogen and progesterone receptor (ER, PR) are the only used predictors of Tam response. However, 25% of ER+/PR+ tumors, 66% of ER+/PR− tumors, and 55% of ER−/PR+ tumors fail to Tam treatment^{1, 2}. The mechanisms responsible for these treatment failures still remain unclear, indicating that it is necessary to characterize the molecular actors involved in BC etiology and resistance that will help to improve BC phenotyping and treatment efficacy and to develop new anticancer compounds and biomarkers.

Major findings recently highlight that sterol metabolism can produce new targets for cancer progression and resistance²⁻⁶. Consistent with these results, we characterized a new pathway in cholesterol metabolism involved in the control of cell differentiation and growth and showed that it is deregulated in breast cancers at the level of Cholesterol Epoxide Hydrolase (ChEH) metabolism⁶⁻⁸. ChEH catalyses selectively the hydrolysis of cholesterol 5,6-epoxides α and β (α-EC and β-EC) into 5α-cholestan-3β,5,6β-triol (CT)^{3, 9, 10} and it is the target of anti-cancer compounds such as Tam and Dendrogenin A^{3, 7, 10-12}. Interestingly, mucin1, a glycoprotein aberrantly overexpressed in numerous cancers, induces a lipid and sterol metabolism transcriptional signature in breast cancer tissue that is predictive of resistance to Tam treatment and is associated with an increase risk of subject death². Among the genes over-expressed are the one coding 7-dehydrocholesterol reductase (DHCR7) one of the subunit of the ChEH¹⁰, suggesting that deregulations at the level of ChEH metabolism may lead to BC progression and resistance to Tam treatment. Consistent with these results, we established that the activity of ChEH in tumor cells generated an unexpected metabolite from CT in cancer cells⁸. We identified the structure of this unknown metabolite as being 6-oxo-cholestan-3β,5α-diol (OCDO), a product of oxidation of CT and characterized that OCDO promotes tumor proliferation and invasion in vitro and in vivo⁸. However the enzyme responsible for the production of OCDO was not identified.

### SUMMARY OF THE INVENTION

The present invention relates to methods for the diagnosis and the treatment of cancer, in particular breast cancer. In particular, the present invention is defined by the claims.

### DETAILED DESCRIPTION OF THE INVENTION

The aim of the inventors was to identify the enzymes involved in the production of OCDO from CT, to determine their role in cancer promotion/invasion and to study the expression of the enzymes regulating OCDO production in BC subject samples versus matched normal tissue. The inventors thus demonstrate here that the interconversion of CT/OCDO is mediated by the enzymes 11β-hydroxysteroid dehydrogenase of type 1 and 2 (11βHSD2 and 11βHSD1). These enzymes are known to regulate the interconversion of cortisol/cortisone^{13, 14}. Importantly, 11βHSD2 was shown involved in tumor cell proliferation and invasion through OCDO production and 11βHSD1 in the reversion of these events through the transformation of OCDO into CT. Moreover, the inventors found that the expression of the enzymes involved in OCDO production are increased in human breast tumors compared with normal tissue samples and overall the histological studies reveal that the enzymatic equilibrium between 11βHSD2 and 11βHSD1 is shifted toward the production of OCDO in tumors. Together this study highlights new functions for the enzyme 11βHSD1 and 11-βHSD2 in cancer progression and as new markers of cancer.

### Diagnostic Methods of the Invention

Accordingly, an object of the present invention relates to a method of diagnosing cancer in a subject comprising the steps of i) determining the expression level of 11βHSD1 and/or 11βHSD2 in a sample obtained from the subject, ii) comparing the expression level determined at step i) with its predetermined reference value and ii) concluding that the subject suffers from a cancer when the expression level of 11βHSD1 is lower than its predetermined reference value or when the expression level of 11βHSD2 is higher than its predetermined reference value.

Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). In some embodiments, the cancer is breast cancer.

The term “sample” means any tissue sample derived from the subject. Said tissue sample is obtained for the purpose of the in vitro evaluation. The sample can be fresh, frozen, fixed (e.g., formalin fixed), or embedded (e.g., paraffin embedded). In some embodiments the sample is a tumor sample. In some embodiments the tumor sample may result from a tumor resected from the subject. In some embodiments, the tumor sample may result from a biopsy performed in a primary tumour of the subject or performed in metastatic sample distant from the primary tumor of the subject. For example an endoscopical biopsy performed in the bowel of the subject affected by a colorectal cancer.

As used herein, the terms “11βHSD1” and “11βHSD2” have their general meaning in the art and refer to the 11β-hydroxysteroid dehydrogenase of type 1 and 2 respectively. Exemplary amino acid sequences for 11βHSD1 and 11βHSD2 are SEQ ID NO: 1 and SEQ ID NO: 2 respectively. Exemplary nucleic acid sequences for 11βHSD1 and 11βHSD2 are SEQ ID NO: 3 and SEQ ID NO: 4 respectively. More particularly, the term “11β-HSD1” as used herein, refers to the 11-beta-hydroxysteroid dehydrogenase type 1 enzyme, variant, or isoform thereof. 11β-HSD1 variants include proteins substantially homologous to native 11β-HSD1, i.e., proteins having one or more naturally or non-naturally occurring amino acid deletions, insertions or substitutions (e.g., 11β-HSD1 derivatives, homologs and fragments). The amino acid sequence of a 11β-HSD1 variant can be at least about 80% identical to a native 11β-HSD1, or at least about 90% identical, or at least about 95% identical with SEQ ID NO: 1. 11β-HSD2 variants include proteins substantially homologous to native 11β-HSD2, i.e., proteins having one or more naturally or non-naturally occurring amino acid deletions, insertions or substitutions (e.g., 11β-HSD2 derivatives, homologs and fragments). The amino acid sequence of a 11β-HSD2 variant can be at least about 80% identical to a native 11β-HSD2, or at least about 90% identical, or at least about 95% identical with SEQ ID NO: 2.

SEQ ID NO: 1: 11βHSD1_homo sapiens
mafmkkyllp ilglfmayyy ysaneefrpe mlqgkkvivt gaskgigrem
ayhlakmgah vvvtarsket lqkvvshcle lgaasahyia gtmedmtfae
qfvagagklm ggldmlilnh itntslnlfh ddihhvrksm evnflsyvvl
tvaalpmlkq sngsivvvss lagkvaypmv aaysaskfal dgffssirke
ysysrvnvsi ticvlglidt etamkaysgi vhmqaapkee caleiikgga
lrgeevyyds slwttllirn perkilefly stsynmdrfi nk
SEQ ID NO: 2: 11βHSD2_homo sapiens
merwpwpsgg awllvaaral lql1rsdlrl grpllaalal laaldwlcqr
llpppaalav laaagwials rlarpqrlpv atravlitgc dsgfgketak
kldsmgftvl atvlelnspg aielrtccsp rlrllqmdlt kpgdisrvle
ftkahttstg lwglvnnagh nevvadaels pvatfrscme vnffgalelt
kgllpllrss rgrivtvgsp agdmpypclg aygtskaava 11mdtfscel
1pwgvkvsii qpgcfktesv rnvgqwekrk qlllanlpqe llqaygkdyi
ehlhgqflhs lrlamsdltp vvdaitdall aarprrryyp gqglglmyfi
hyylpeglrr rflqaffish clpralqpgq pgttppgdaa qdpnlspgps
pavar
SEQ ID NO: 3: 11βHSD1_homo sapiens
gggaaattgg ctagcactgc ctgagactac tccagcctcc cccgtccctg
atgtcacaat tcagaggctg ctgcctgctt aggaggttgt agaaagctct
gtaggttctc tctgtgtgtc ctacaggagt cttcaggcca gctccctgtc
ggatggcttt tatgaaaaaa tatctcctcc ccattctggg gctcttcatg
gcctactact actattctgc aaacgaggaa ttcagaccag agatgctcca
aggaaagaaa gtgattgtca caggggccag caaagggatc ggaagagaga
tggcttatca tctggcgaag atgggagccc atgtggtggt gacagcgagg
tcaaaagaaa ctctacagaa ggtggtatcc cactgcctgg agcttggagc
agcctcagca cactacattg ctggcaccat ggaagacatg accttcgcag
agcaatttgt tgcccaagca ggaaagctca tgggaggact agacatgctc
attctcaacc acatcaccaa cacttctttg aatctttttc atgatgatat
tcaccatgtg cgcaaaagca tggaagtcaa cttcctcagt tacgtggtcc
tgactgtagc tgccttgccc atgctgaagc agagcaatgg aagcattgtt
gtcgtctcct ctctggctgg gaaagtggct tatccaatgg ttgctgccta
ttctgcaagc aagtttgctt tggatgggtt cttctcctcc atcagaaagg
aatattcagt gtccagggtc aatgtatcaa tcactctctg tgttcttggc
ctcatagaca cagaaacagc catgaaggca gtttctggga tagtccatat
gcaagcagct ccaaaggagg aatgtgccct ggagatcatc aaagggggag
ctctgcgcca agaagaagtg tattatgaca gctcactctg gaccactctt
ctgatcagaa atccatgcag gaagatcctg gaatttctct actcaacgag
ctataatatg gacagattca taaacaagta ggaactccct gagggctggg
catgctgagg gattttggga ctgttctgtc tcatgtttat ctgagctctt
atctatgaag acatcttccc agagtgtccc cagagacatg caagtcatgg
gtcacacctg acaaatggaa ggagttcctc taacatttgc aaaatggaaa
tgtaataata atgaatgtca tgcaccgctg cagccagcag ttgtaaaatt
gttagtaaac ataggtataa ttaccagata gttatattaa atttatatct
tatatataat aatatgtgat gattaataca atattaatta taataaaggt
cacataaact ttataaattc ataactggta gctataactt gagcttattc
aggatggttt ctttaaaacc ataaactgta caaatgaaat ttttcaatat
ttgtttctta aaaaaaaaaa aaaaaaa
SEQ ID NO: 4: 11βHSEC_homo sapiens
ccctctcgcg ccccaggccg gtgtaccccc gcactccgcg ccccggccta
gaagctctct ctccccgctc cccggcccgg cccccgcccc gccccgcccc
agcccgctgg gccgccatgg agcgctggcc ttggccgtcg ggcggcgcct
ggctgctcgt ggctgcccgc gcgctgctgc agctgctgcg ctcagacctg
cgtctgggcc gcccgctgct ggcggcgctg gcgctgctgg ccgcgctcga
ctggctgtgc cagcgcctgc tgcccccgcc ggccgcactc gccgtgctgg
ccgccgccgg ctggatcgcg ttgtcccgcc tggcgcgccc gcagcgcctg
ccggtggcca ctcgcgcggt gctcatcacc ggctgtgact ctggttttgg
caaggagacg gccaagaaac tggactccat gggcttcacg gtgctggcca
ccgtattgga gttgaacagc cccggtgcca tcgagctgcg tacctgctgc
tcccctcgcc taaggctgct gcagatggac ctgaccaaac caggagacat
tagccgcgtg ctagagttca ccaaggccca caccaccagc accggcctgt
ggggcctcgt caacaacgca ggccacaatg aagtagttgc tgatgcggag
ctgtctccag tggccacttt ccgtagctgc atggaggtga atttctttgg
cgcgctcgag ctgaccaagg gcctcctgcc cctgctgcgc agctcaaggg
gccgcatcgt gactgtgggg agcccagcgg gggacatgcc atatccgtgc
ttgggggcct atggaacctc caaagcggcc gtggcgctac tcatggacac
attcagctgt gaactccttc cctggggggt caaggtcagc atcatccagc
ctggctgctt caagacagag tcagtgagaa acgtgggtca gtgggaaaag
cgcaagcaat tgctgctggc caacctgcct caagagctgc tgcaggccta
cggcaaggac tacatcgagc acttgcatgg gcagttcctg cactcgctac
gcctggccat gtccgacctc accccagttg tagatgccat cacagatgcg
ctgctggcag ctcggccccg ccgccgctat taccccggcc agggcctggg
gctcatgtac ttcatccact actacctgcc tgaaggcctg cggcgccgct
tcctgcaggc cttcttcatc agtcactgtc tgcctcgagc actgcagcct
ggccagcctg gcactacccc accacaggac gcagcccagg acccaaacct
gagccccggc ccttccccag cagtggctcg gtgagccatg tgcacctatg
gcccagccac tgcagcacag gaggctccgt gagcccttgg ttcctccccg
aaaaccccca gcattacgat cccccaagtg tcctggaccc tggcctaaag
aatcccaccc ccacttcatg cccactgccg atgcccaatc caggcccggt
gaggccaagg tttcccagtg agcctctgcg cctctccact gtttcatgag
cccaaacacc ctcctggcac aacgctctac cctgcagctt ggagaactcc
gctggatggg gagtctcatg caagacttca ctgcagcctt tcacaggact
ctgcagatag tgcctctgca aactaaggag tgactaggtg ggttggggac
cccctcagga ttgtttctcg gcaccagtgc ctcagtgctg caattgaggg
ctaaatccca agtgtctctt gactggctca agaattaggg ccccaactac
acacccccaa gccacaggga agcatgtact gtacttccca attgccacat
tttaaataaa gacaaatttt tatttcttct aaaaaaaaaa aaaaaaaaaa
aaaaaaaaaa aaaaaaaaaa aaaaaaaaaa aaaaaaaaa

A further object of the present invention relates to a method for determining the survival time of subject suffering from a cancer comprising the steps of i) determining the expression level of 11βHSD1 and/or 11βHSD2 in a tumor sample obtained from the subject, ii) comparing the expression level determined at step i) with its predetermined reference value and ii) concluding that the subject will have a long survival time when the expression level of 11βHSD1 is higher than its predetermined reference value or concluding that the subject will have a short survival time when the expression level of 11βHSD2 is lower than its predetermined reference value.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

A further object of the present invention relates to a method for determining whether a subject suffering from a cancer will achieve a response with tamoxifen or dendrogenin A of i) determining the expression level of 11βHSD1 and/or 11βHSD2 in a tumor sample obtained from the subject, ii) comparing the expression level determined at step i) with its predetermined reference value and ii) concluding that the subject will achieve a response with tamoxifen or dendrogenin A when the expression level of 11βHSD1 is higher than its predetermined reference value or when the expression level of 11βHSD2 is lower than its predetermined reference value.

As used herein, the term, “tamoxifen” has its general meaning in the art and refers to an antagonist of the estrogen receptor in breast tissue via its active metabolite, hydroxytamoxifen. More particularly, the term “tamoxifen” refers to (Z)-2-[4-(1,2-diphenylbut-1-enyl)phenoxy]-N,N-dimethylethanamine or a salt thereof.

As used herein, the term “Dendrogenin A” refers to the pharmaceutically active compound 5-hydroxy-6-[2-(1H-imidazol-4-yl)ethylamino]cholestan-3-ol. Dendrogenin A is disclosed in WO03/89449 and de Medina et al (J. Med. Chem., 2009) as free base. Its structural formula is the following:

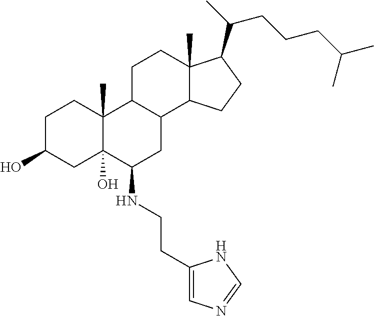 [SMILES: CC(C)CCCC(C)[C@H]1CCC2C3C[C@@H](NCCc4cnc[nH]4)[C@@]4(O)C[C@@H](O)CC[C@]4(C)C3CC[C@@]21C]

Measuring the expression level of a gene (i.e. 1βHSD1 and/or 11βHSD2) can be performed by a variety of techniques well known in the art.

In some embodiments, the expression level is determined at nucleic acid level. Typically, the expression level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dichlorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOTTM (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as ³H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, coumarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute expression level or normalized expression level. Typically, expression levels are normalized by correcting the absolute expression level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the expression level in one sample, e.g., a subject sample, to another sample, or between samples from different sources.

In some embodiments, the expression level of 11βHSD1 and/or 11βHSD2 is determined at the protein level by any well known method in the art. Typically, such methods comprise contacting the tissue sample with at least one selective binding agent capable of selectively interacting with 11βHSD1 and/or 11βHSD2. The selective binding agent may be polyclonal antibody or monoclonal antibody, an antibody fragment, synthetic antibodies, or other protein-specific agents such as nucleic acid or peptide aptamers. Several antibodies have been described in the prior art and many antibodies are also commercially available such as described in the EXAMPLE. For the detection of the antibody that makes the presence of the 11βHSD1 and/or 11βHSD2 detectable by microscopy or an automated analysis system, the antibodies may be tagged directly with detectable labels such as enzymes, chromogens or fluorescent probes or indirectly detected with a secondary antibody conjugated with detectable labels. The preferred method according to the present invention is immunohistochemistry. Immunohistochemistry typically includes the following steps:
fixing said sample with formalin,
embedding said sample in paraffin.
cutting said sample into sections for staining
incubating said sections with the binding partner specific for
rinsing said sections
incubating said section with a biotinylated secondary antibody
revealing the antigen-antibody complex with avidin-biotin-peroxidase complex
   Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of 11βHSD1 and/or 11βHSD2 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the levels of the cytokines in a group of reference, one can use algorithmic analysis for the statistic treatment of the measured concentrations of cytokines in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

A predetermined reference value can be relative to a number or value derived from population studies, including without limitation, subjects of the same or similar age range, subjects in the same or similar ethnic group, and subjects having the same severity of cancer. Such predetermined reference values can be derived from statistical analyses and/or risk prediction data of populations obtained from mathematical algorithms and computed indices. In some embodiments, the predetermined reference values are derived from the expression level of 11βHSD1 and/or 11βHSD2 in a control sample derived from one or more subjects who do not suffer from cancer. Furthermore, retrospective measurement of the level of the selected biomarker in properly banked historical subject samples may be used in establishing these predetermined reference values.

In some embodiments, the predetermined reference value is correlated the survival time (e.g. disease-free survival (DFS) and/or the overall survival (OS)). Accordingly, the predetermined reference value may be typically determined by carrying out a method comprising the steps of

a) providing a collection of tumor samples from subject suffering from the same cancer;

b) providing, for each tumor sample provided at step a), information relating to the actual clinical outcome for the corresponding subject (i.e. the duration of the disease-free survival (DFS) and/or the overall survival (OS));

c) providing a serial of arbitrary quantification values;

d) determining the level of the selected biomarker (e.g. 11βHSD1 or 11βHSD2) for each tumor sample contained in the collection provided at step a);

e) classifying said tumor samples in two groups for one specific arbitrary quantification value provided at step c), respectively: (i) a first group comprising tumor samples that exhibit a quantification value for level that is lower than the said arbitrary quantification value contained in the said serial of quantification values; (ii) a second group comprising tumor samples that exhibit a quantification value for said level that is higher than the said arbitrary quantification value contained in the said serial of quantification values; whereby two groups of tumor samples are obtained for the said specific quantification value, wherein the tumor samples of each group are separately enumerated;

f) calculating the statistical significance between (i) the quantification value obtained at step e) and (ii) the actual clinical outcome of the subjects from which tumor samples contained in the first and second groups defined at step derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the expression level of the selected biomarker (e.g. 11βHSD1 or 11βHSD2) has been assessed for 100 tumor samples of 100 subjects. The 100 samples are ranked according to the expression level of the selected biomarker (e.g. 11βHSD1 or 11βHSD2). Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of the selected biomarker (e.g. 11βHSD1 or 11βHSD2) corresponding to the boundary between both subsets for which the p value is minimum is considered as the predetermined reference value. It should be noted that the predetermined reference value is not necessarily the median value of levels of the selected biomarker (e.g. 11βHSD1 or 11βHSD2).

Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above.

For example, according to this specific embodiment of a “cut-off” value, the outcome can be determined by comparing the expression level of the selected biomarker (e.g. 11βHSD1 or 11βHSD2) with the range of values which are identified. In certain embodiments, a cut-off value thus consists of a range of quantification values, e.g. centered on the quantification value for which the highest statistical significance value is found (e.g. generally the minimum p value which is found). For example, on a hypothetical scale of I to 10, if the ideal cut-off value (the value with the highest statistical significance) is 5, a suitable (exemplary) range may be from 4-6. For example, a subject may be assessed by comparing values obtained by measuring the expression level of 11βHSD2, where values greater than 5 reveal a poor prognosis and values less than 5 reveal a good prognosis. In a another embodiment, a subject may be assessed by comparing values obtained by measuring the expression level of 11βHSD2 and comparing the values on a scale, where values above the range of 4-6 indicate a poor prognosis and values below the range of 4-6 indicate a good prognosis, with values falling within the range of 4-6 indicating an intermediate occurrence (or prognosis).

### Therapeutic Methods of the Invention

Once the subject is diagnosed as suffering from cancer, the physician can take the choice to administer the subject with the most accurate treatment. Typically, the treatment includes chemotherapy, radiotherapy, and immunotherapy.

In some embodiments, the subject once diagnosed as suffering from cancer by the method of the invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridin A and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the subject once diagnosed as suffering from cancer is administered with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S. Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S. Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the subject once diagnosed as suffering from a cancer is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erythropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference].

The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

In some embodiments, the subject once diagnosed as suffering from cancer is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In some embodiments, when it is determined that the subject will achieve a response with tamoxifen or dendrogenin A, the subject is then administered with said drugs.

In some embodiments, the subject once diagnosed as suffering from cancer is administered with a 11β-HSD2 inhibitor.

The term “11β-HSD2 inhibitor” includes any agents which inhibit or decrease the activity or expression of 11β-HSD2.

In some embodiments, the 11β-HSD2 inhibitor is a small molecule, such as a steroid or a derivative thereof. In some embodiments, the steroid is 3α, 5α-reduced. Examples of 11β-HSD2 inhibitors include, but are not limited to, 3α, 5α-reduced-11β-OH-progesterone, 3α, 5α-reduced-11β-OH-testosterone, 3α, 5α-reduced-11β-OH-androstenedione, 3α, 5α-reduced-11-keto-progesterone, 3α, 5α-reduced-11-dehydro-corticosterone, 3α, 5α-reduced-corticosterone, 3α, 5α-reduced-11β-OH-pregnenolone, 3α, 5α-reduced-11β-OH-dehydro-epiandrostenedione, 3α, 5α-reduced-pregnenolone, 3α, 5α-reduced-dehydro-epiandrostenedione, 3α, 5α-reduced aldosterone, and 3α, 5α-reduced deoxycorticosterone. Other examples of 11β-HSD2 inhibitors include 11β-OH-progesterone, 11β-OH-pregnenolone, 11β-OH-dehydro-epiandrostenedione, 11β-OH-testosterone, 11-keto-progesterone, 5α-dihydro-corticosterone, 3α, 5α-reduced deoxy-corticosterone, glycyrrhetinic acid or carbenoxolone.

Other examples of 11β-HSD2 inhibitor include the compound disclosed in U.S. Pat. No. 7,659,287, in particular the compounds having, the formula of:

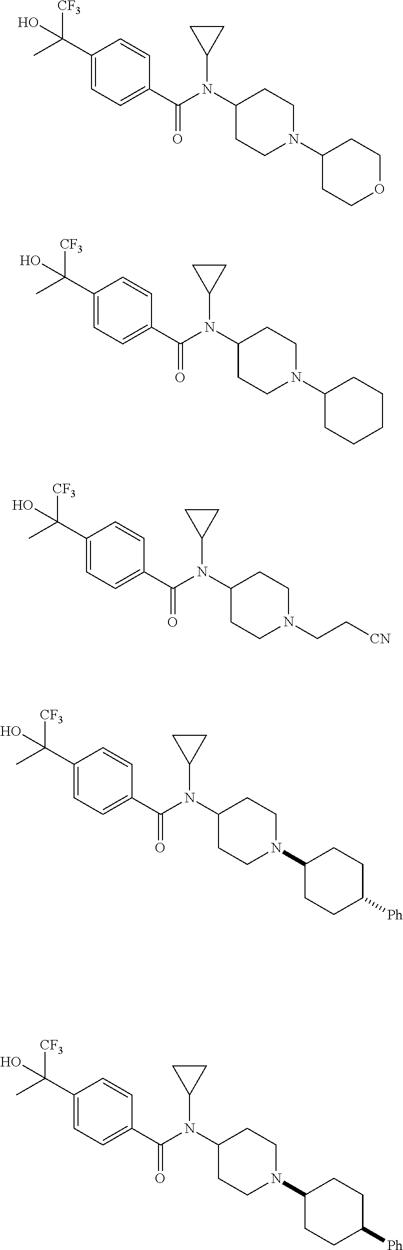

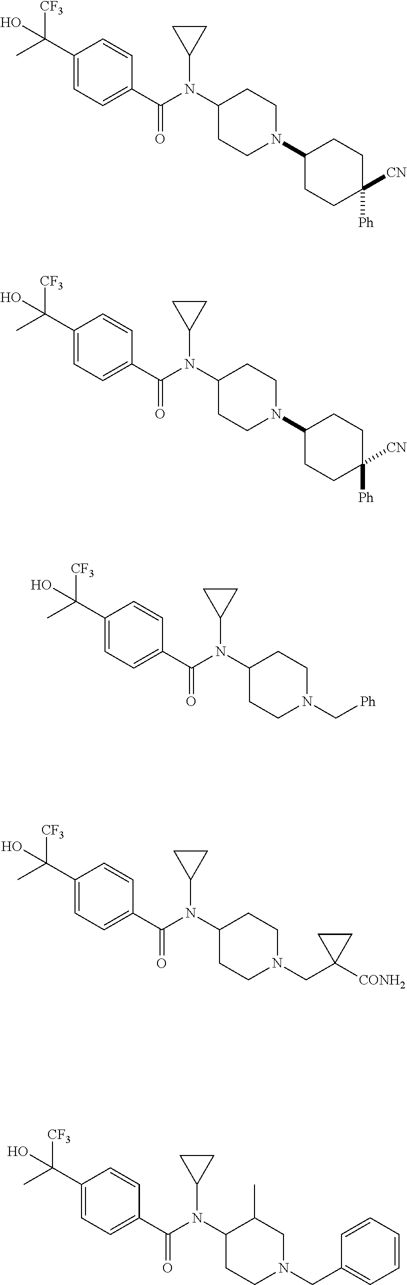

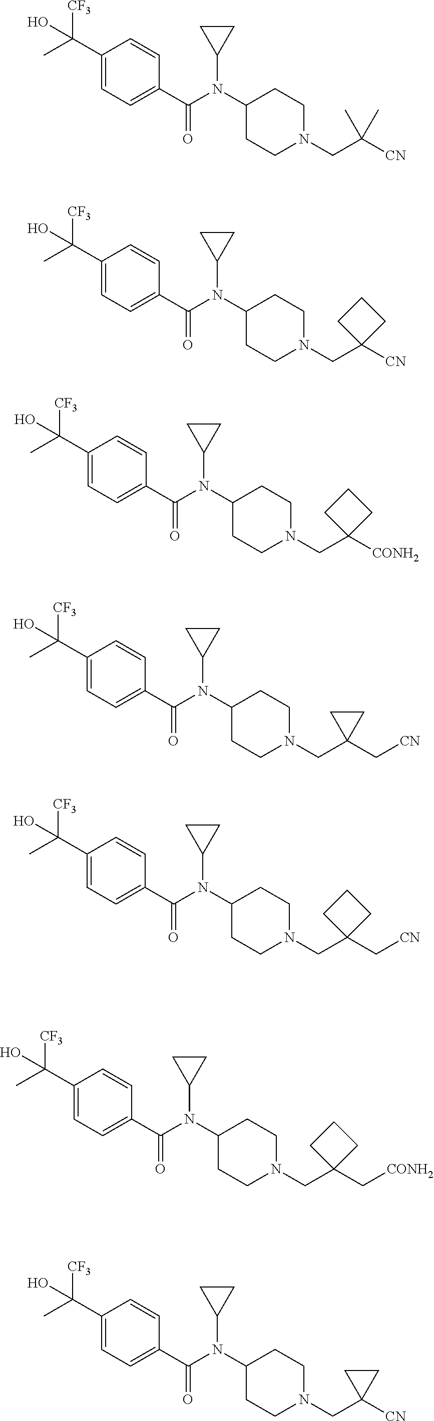

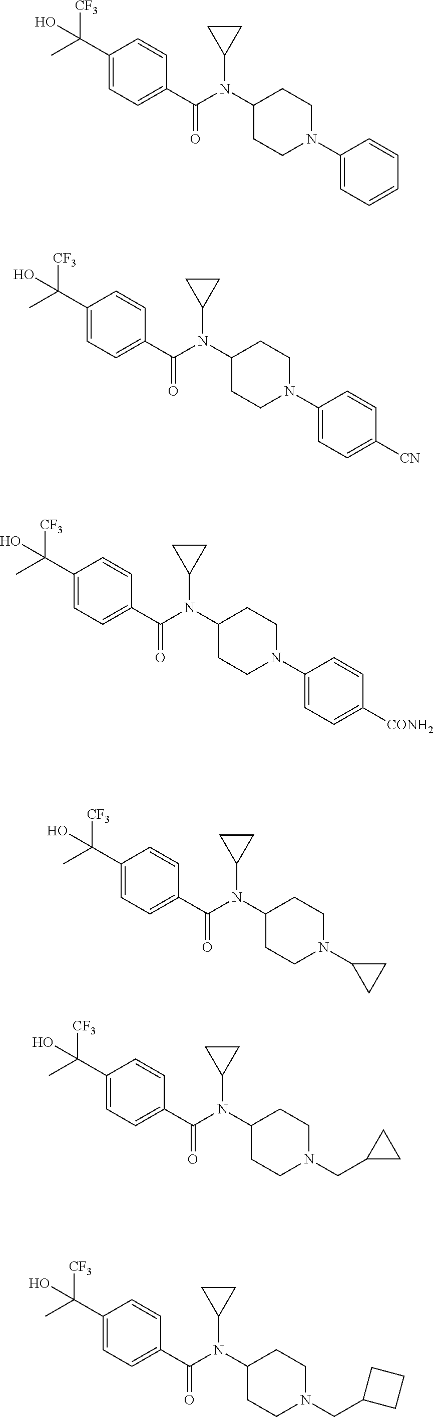 [SMILES: CC(O)(c1ccc(C(=O)N(C2CC2)C2CCN(c3ccccc3)CC2)cc1)C(F)(F)F ; CC(O)(c1ccc(C(=O)N(C2CC2)C2CCN(c3ccc(C#N)cc3)CC2)cc1)C(F)(F)F ; CC(O)(c1ccc(C(=O)N(C2CC2)C2CCN(c3ccc(C(N)=O)cc3)CC2)cc1)C(F)(F)F ; CC(O)(c1ccc(C(=O)N(C2CC2)C2CCN(C3CC3)CC2)cc1)C(F)(F)F ; CC(O)(c1ccc(C(=O)N(C2CC2)C2CCN(CC3CC3)CC2)cc1)C(F)(F)F ; CC(O)(c1ccc(C(=O)N(C2CC2)C2CCN(CC3CCC3)CC2)cc1)C(F)(F)F]

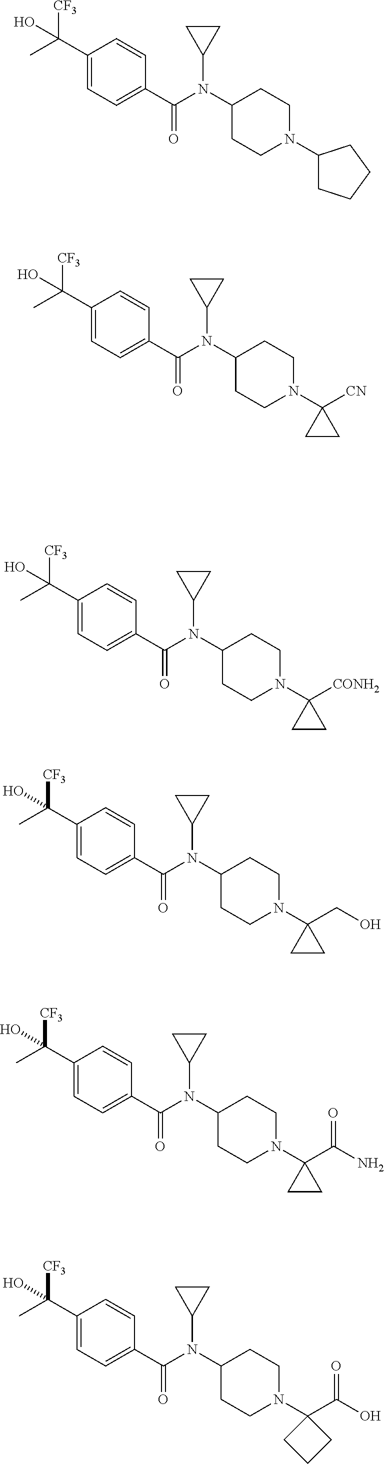 [SMILES: CC(O)(c1ccc(C(=O)N(C2CC2)C2CCN(C3CCCC3)CC2)cc1)C(F)(F)F ; CC(O)(c1ccc(C(=O)N(C2CC2)C2CCN(C3(C#N)CC3)CC2)cc1)C(F)(F)F ; CC(O)(c1ccc(C(=O)N(C2CC2)C2CCN(C3(C(N)=O)CC3)CC2)cc1)C(F)(F)F ; C[C@](O)(c1ccc(C(=O)N(C2CC2)C2CCN(C3(CO)CC3)CC2)cc1)C(F)(F)F ; C[C@](O)(c1ccc(C(=O)N(C2CC2)C2CCN(C3(C(N)=O)CC3)CC2)cc1)C(F)(F)F ; C[C@](O)(c1ccc(C(=O)N(C2CC2)C2CCN(C3(C(=O)O)CCC3)CC2)cc1)C(F)(F)F]

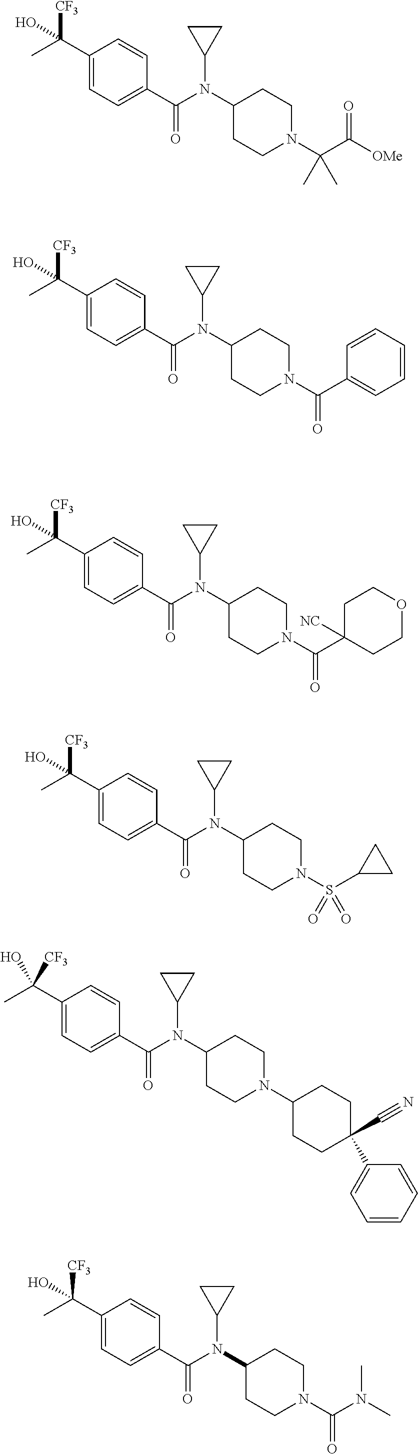

Other examples of 11β-HSD2 inhibitor include the compound disclosed in U.S. Pat. No. 7,495,012, in particular the compounds having the formula of:
syn-2,6-dimethyl-1-(4-(2,2,2-trifluoro-1-hydroxy-1-methylethyl)phenyl-sulfonyl)-piperidine,
2-(R)-2-methyl-1-(4-(2,2,2-trifluoro-1-hydroxy-1-methylethyl)phenyl-sulfonyl)-piperidine,
2-(S)-2-methyl-1-(4-(2,2,2-trifluoro-1-hydroxy-1-methylethyl)phenyl-sulfonyl)-piperidine,
2-ethyl-1-(4-(2,2,2-trifluoro-1-hydroxy-1-methylethyl)phenylsulfonyl)-piperidine,
1-(4-(2,2,2-trifluoro-1-hydroxy-1-methylethyl)phenylsulfonyl)-piperidine,
2-(4-(2,2,2-trifluoro-1-hydroxy-1-methylethyl)phenylsulfonyl)-1,2,3,4-tetrahydroisoquinoline,
2-(S)-2-(pyridin-3-yl)-1-(4-(2,2,2-trifluoro-1-hydroxy-1-methylethyl)-phenylsulfonyl)-piperidine,
1-(4-(2,2,2-trifluoro-1-hydroxy-1-methylethyl)phenylsulfonyl)-1,2,3,4-tetrahydroquinoline,
3-fluoro-1-(4-(2,2,2-trifluoro-1-hydroxy-1-methylethyl)phenylsulfonyl)-piperidine,
1-(4-(2,2,2-trifluoro-1-hydroxy-1-methylethyl)phenylsulfonyl)-2-(2-imidazol-1-yl-ethyl)piperidine,
2-(2-pyrazol-1-yl-ethyl)-1-(4-(2,2,2-trifluoro-1-hydroxy-1-methylethyl)-phenylsulfonyl)-piperidine, and
2-(2-hydroxyethyl)-1-(4-(2,2,2-trifluoro-1-hydroxy-1-methylethyl)-phenylsulfonyl)-piperidine.

Other examples of 11β-HSD2 inhibitor include the compound disclosed in U.S. Pat. No. 8,138,190, in particular the compounds having the formula of:

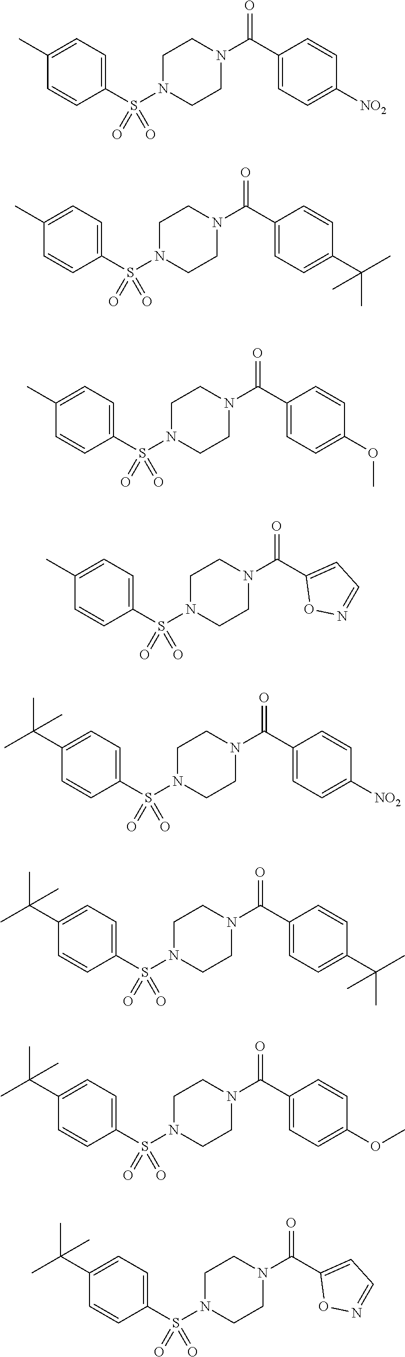

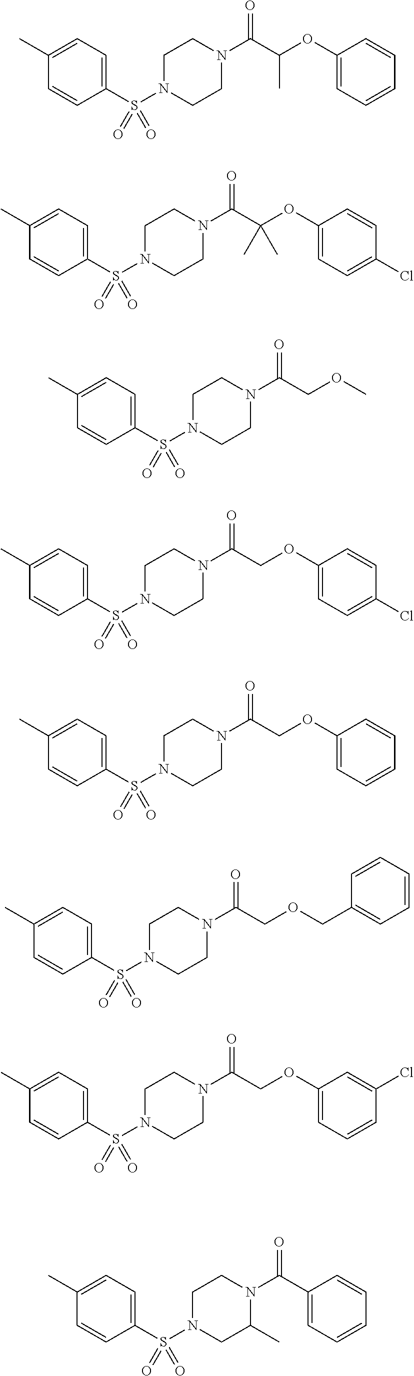

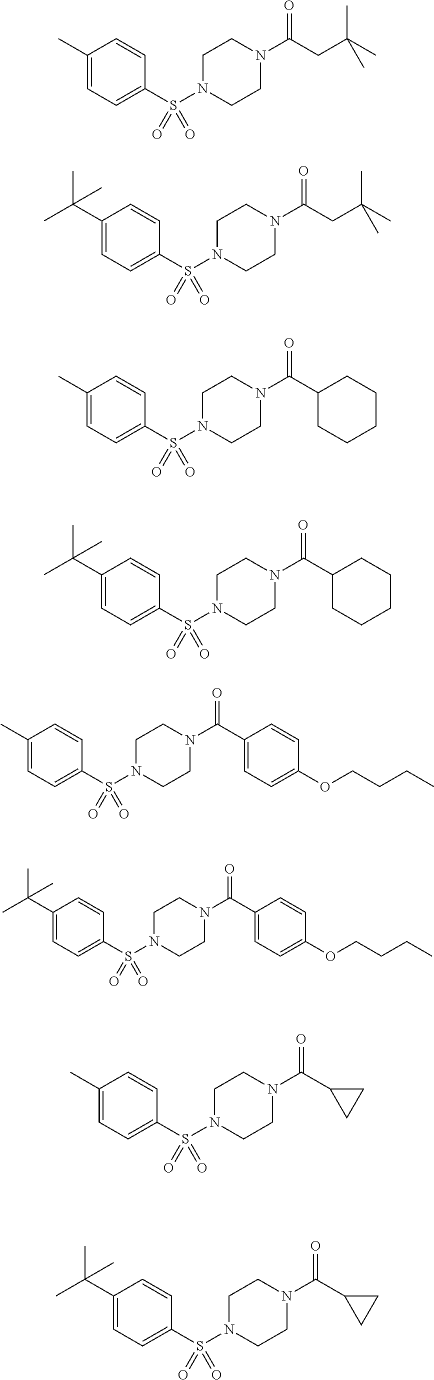

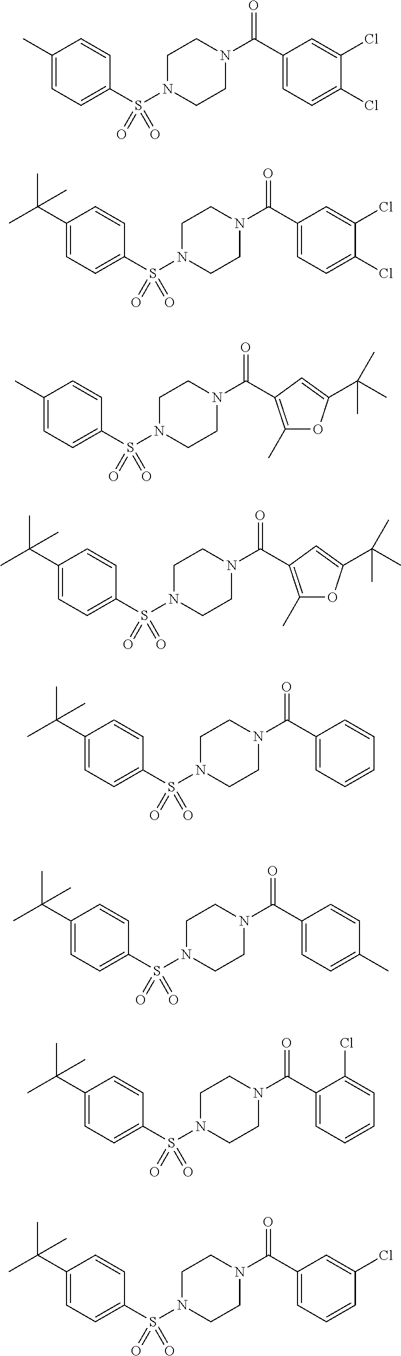

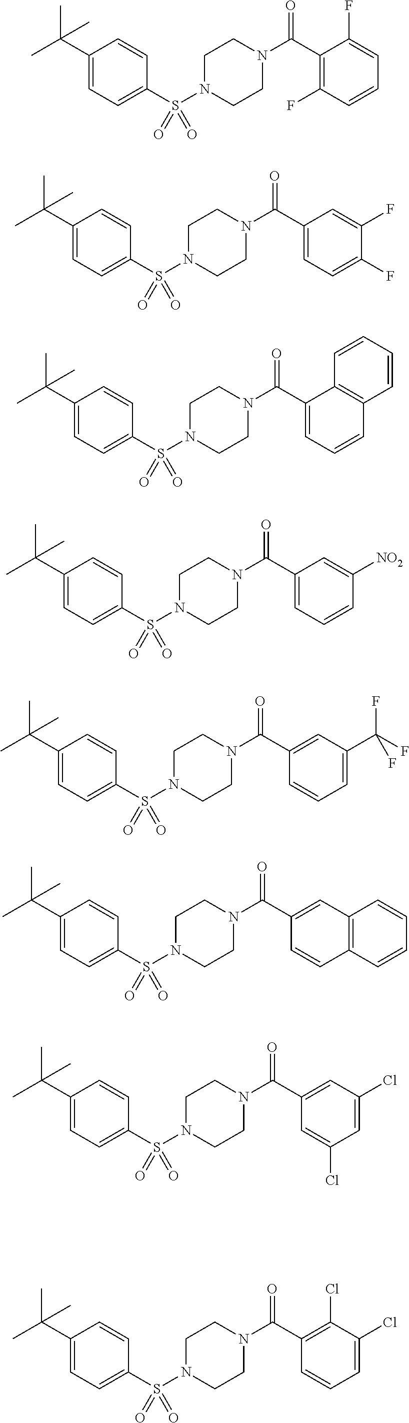

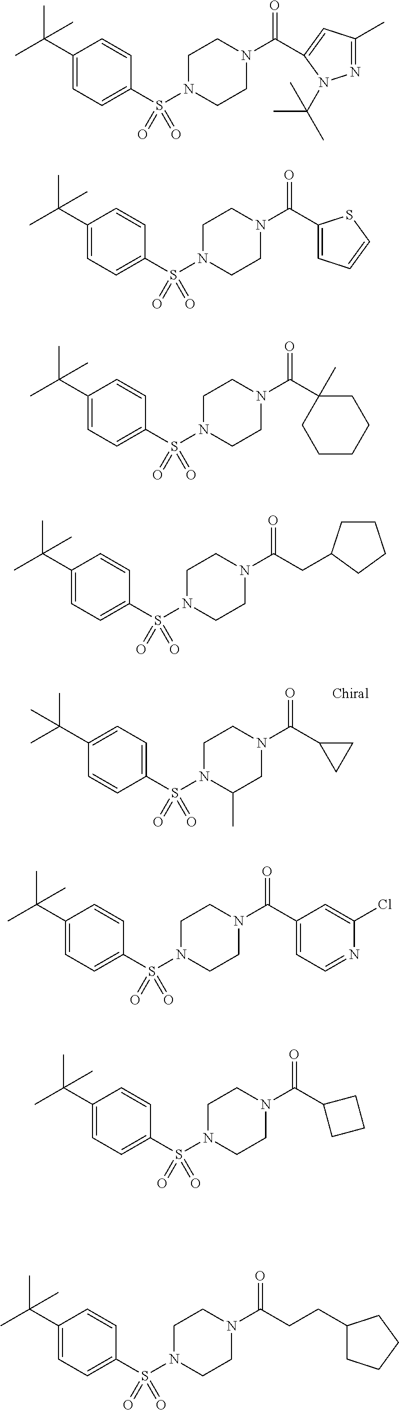

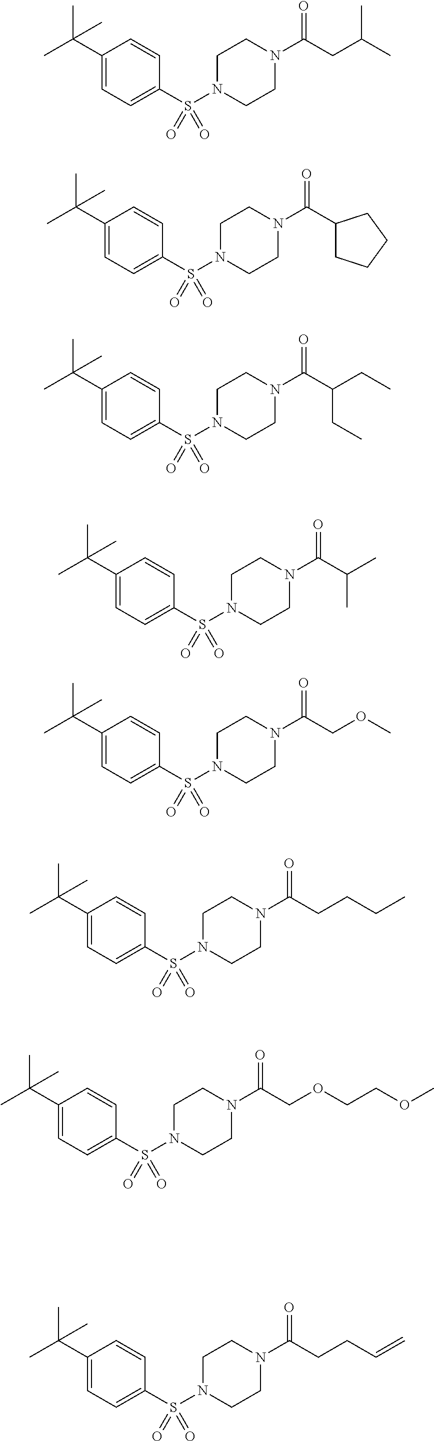

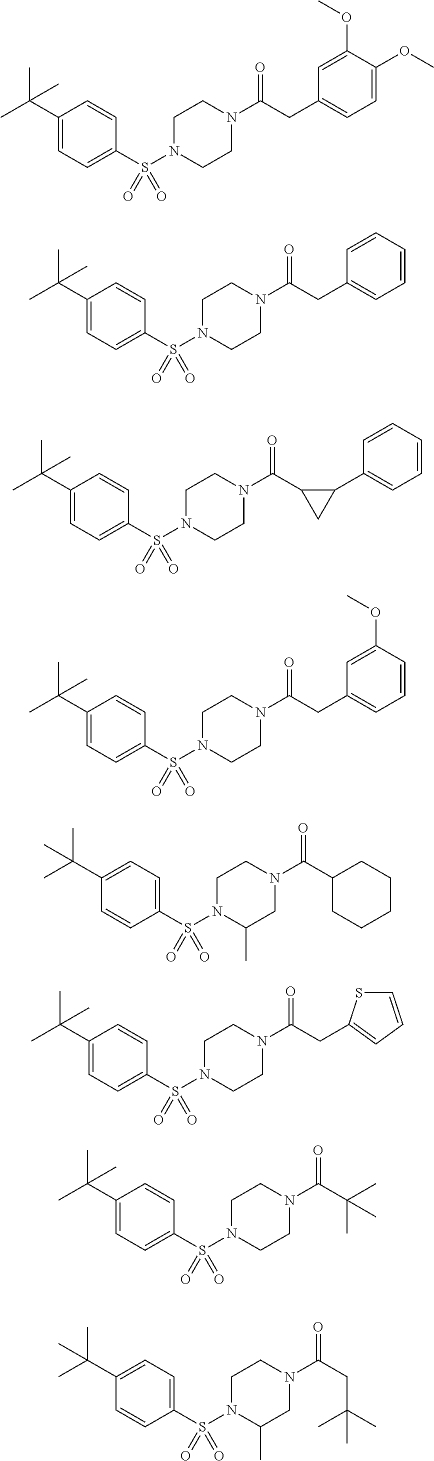

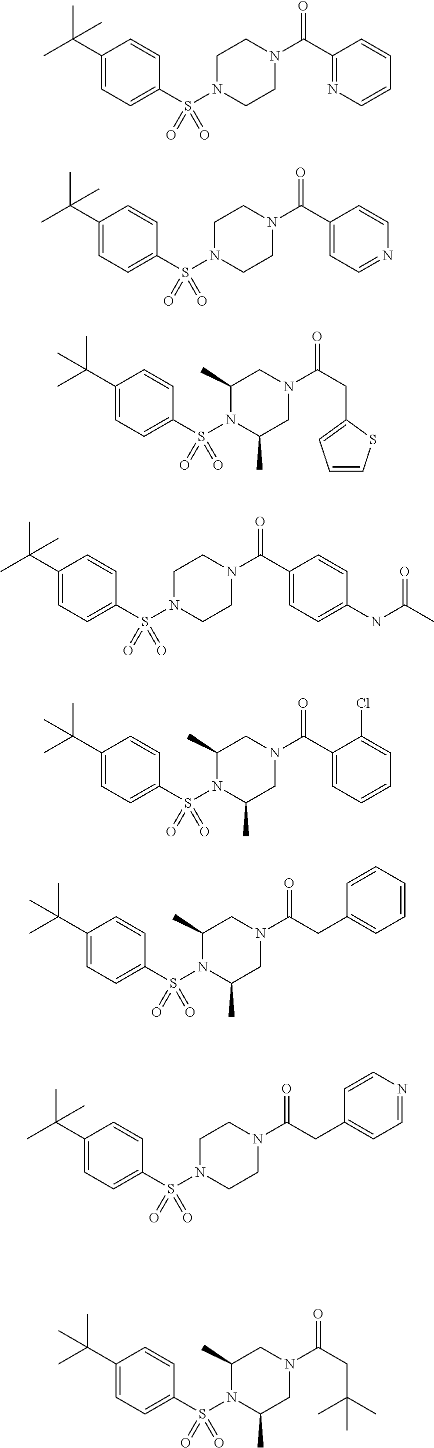

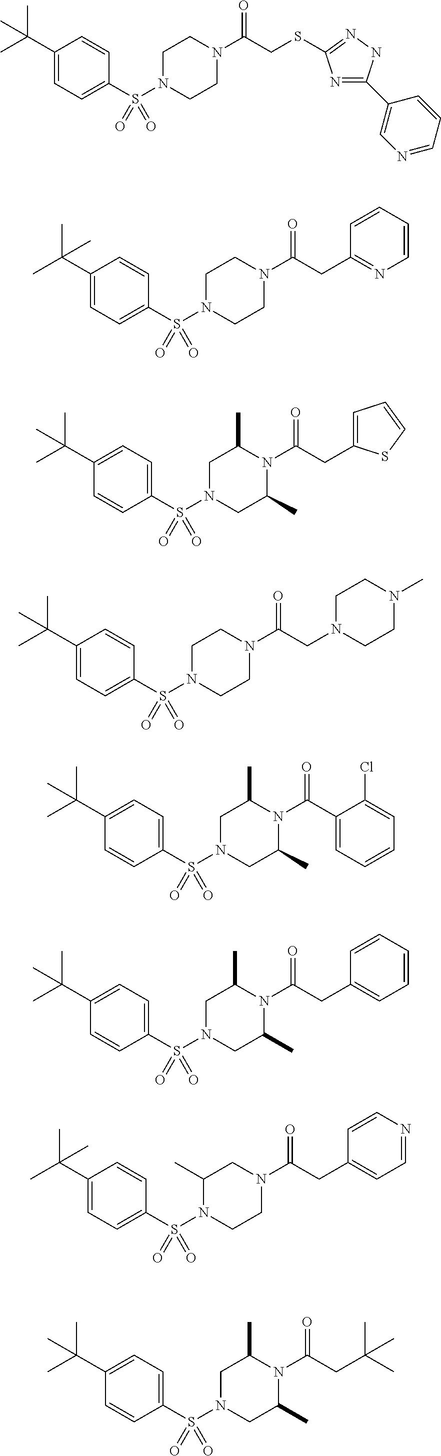

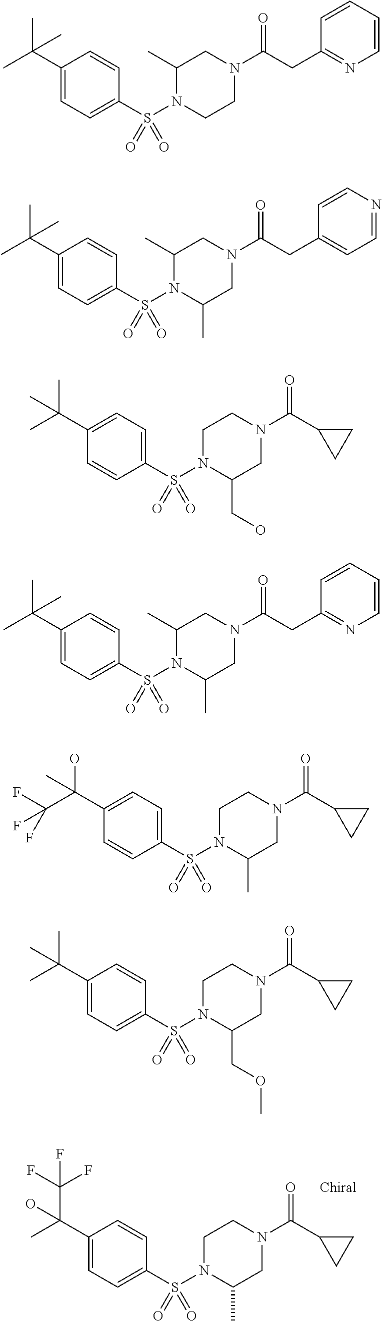

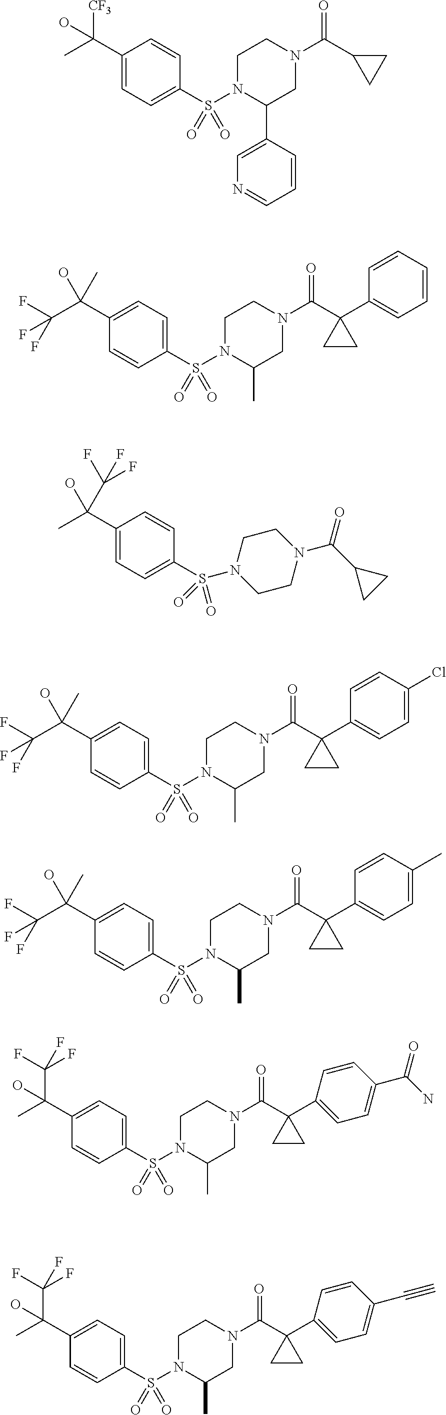

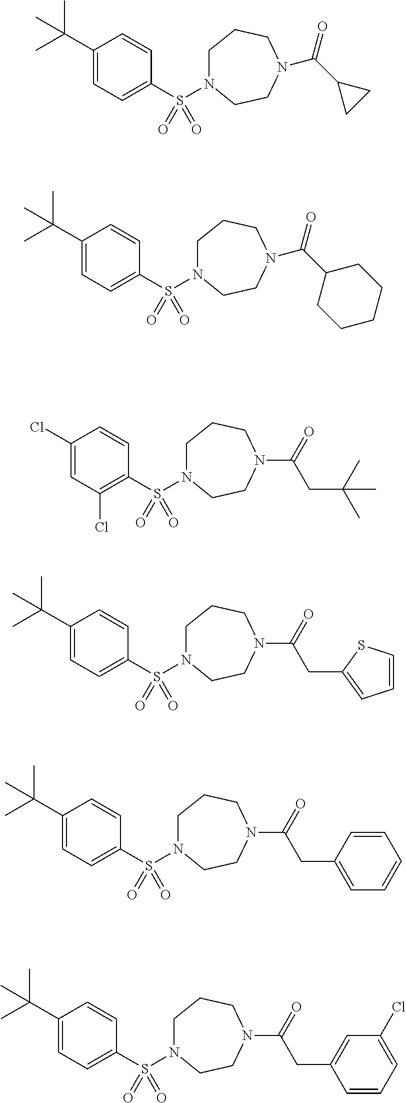

In some embodiments, the 11β-HSD2 inhibitor is an inhibitor of 11β-HSD2 expression.

An “inhibitor of expression” refers to a natural or synthetic compound that has a biological effect to inhibit the expression of a gene.

In some embodiments, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of the targeted mRNA by binding thereto and thus preventing protein translation or increasing mRNA degradation, thus decreasing the level of the targeted protein (i.e. 11β-HSD2), and thus activity, in a cell. For example, antisense oligonucleotides of at least about 15 bases and complementary to unique regions of the mRNA transcript sequence encoding the target protein can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the targeted mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Clifton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In some embodiments, the subject once diagnosed as suffering from cancer is administered with a nucleic acid encoding for 11β-HSD1. Typically, the nucleic acid encoding for 11β-HSD1 is delivered with a vector as described above.

Typically the active ingredient as described above (e.g. tamoxifen, dendrogenin A, inhibitor of 11β-HSD2, nucleic acid encoding for 11β-HSD1 . . . ) is administered to the subject in a therapeutically effective amount.

By a “therapeutically effective amount” of the active ingredient as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the active ingredient is administered to the subject in the form of a pharmaceutical composition. Typically, the active ingredient may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1A-F. OCDO is produced and secreted from MCF7 tumor cells incubated with EC or CT. Representative TLC autoradiograms showing time dependent production of OCDO in MCF7 cells treated with ¹⁴C-αEC (A,B) or ¹⁴C-βEC (C,D) or ¹⁴C-CT (E, F) and quantitative analyses of the metabolites produced in each condition from three separate experiments (±s.e.m). The metabolites extracted from the cells (left panels) or from the medium (right panels) were analyzed by TLC analysis and the region corresponding to radioactive metabolites of interest were recovered and counted using a β-counter.

FIG. 2A-L. OCDO is a tumor promoter in vitro and in vivo and its inhibition contributes to the anti-tumor effects of Tam and DDA. (A, B) Histograms representing the effect of OCDO or 17β-estrogen (E2) on MCF7 (A) and TS/A (B) cell proliferation after 24 h treatment using a colorimetric immunoassay measuring BrDU incorporation in DNA (C, D) Histogram representing the effect of OCDO on MCF7 (C) and TS/A (D) cell invasion. Data are the mean of three separate experiments (±s.e.m), *P<0.05, **P<0.01, ***P<0.001 (Student's t-test). (E, F) Mice were implanted s.c. with MCF7 (E) or TS/A (F) cells and animals (8 per group) were treated s.c. every day starting on the day of implantation with either the solvent vehicle or OCDO (16 μg/kg for MCF7 or 50 μg/kg for TS/A). Animals were monitored for tumour growth twice a week. The data are representative of three independent experiments. The mean tumor volume±s.e.m is shown, *P<0.05, **P<0.01, ***P<0.001 (analysis of variance (ANOVA), Dunnett's post test). (G) Mean (±s.e.m) of Ki67 positive cell number determined from IHC staining of MCF7 tumor sections from (E), n=8, *P<0.05 (Student's t-test) using HistoQuant, Pannoramic Viewer (3DHistech). (H) Representative Ki67 staining of TS/A tumor sections from (F) showing increased staining in OCDO-treated tumor compared with control-treated tumor. (I, J, K). Murine E0771(I), or human MDA-MB231 (J) or MDA-MB468 (K) cells implanted s.c. into mice (8 per group) and animals were treated s.c. with either the solvent vehicle or OCDO (16 μg/kg for MDA-MB-231 and MDA-MB-468 or 50 μg/kg for E0771). The data are representative of two independent experiments. Statistical analysis was performed as in E and F. (L) Mice were implanted s.c. with TS/A cells and animals (8 per group) were treated s.c. every day with either the solvent vehicle, OCDO (50 μg/kg), Tam (56 mg/kg), DDA (20 mg/kg) or the combination of Tam (56 mg/kg)+OCDO (50 μg/kg) or DDA (20 mg/kg)+OCDO (50 μg/kg). The data are representative of three independent experiments. Statistical analysis was performed as in E and F.

FIG. 3. We hypothesized that 11β-HSD type 2 (11HSD2) which catalyzes the dehydrogenation of cortisol into cortisone is the enzyme that produces OCDO from CT, and 11β-HSD type 1 (11HSD1) which catalyzes the hydrogenation of cortisone into cortisol is the enzyme that realizes the reverse reaction (CT production from OCDO). We also hypothesized that H6PDH which produces the cofactor NADPH necessary for the 11βHSD1 reductase activity and the production of cortisol is also necessary for the production of CT.

FIG. 4A-F. 11βHSD2 and 11βHSD1 are the enzymes producing OCDO and CT respectively. HEK-273 cells (5×10⁶ cells) were transfected by electroporation with the plasmids coding either the enzymes 11βHSD2 (HSD2), 11βHSD1 (HSD1), H6PDH, the control empty vector (mock) or were co-transfected with a plasmid coding 11βHSD1 or H6PDH, and analyzed as followed: (A) the expression of 11βHSD2 was confirmed by immunoblotting using a specific antibody against 11βHSD2 and normalized with actin; (B, C) the production of cortisone (B) or OCDO (C) was determined by incubating the mock or the HSD2-transfected cells with ³H-cortisol or ¹⁴C-CT for 8 h at 37° c. respectively. Lipids extracted from the cell and the media were analyzed by TLC analysis and the region corresponding to radioactive metabolites of interest were recovered and counted using a β-counter; (D) the expression of 11βHSD1 and H6PDH was confirmed by immunoblotting using a specific antibody against 11βHSD1 or H6PDH and normalized with actin; (E, F) HEK-273 cells expressing 11βHSD1, H6PDH, both enzymes or the control empty vector (mock) were incubated either with ³H-cortisone (E) or ¹⁴C-OCDO (F) for 72 h and the radioactive metabolites of interest were analyzed as in B and C. The results in B, C, E, F are the mean (±s.e.m) of three experiments, **P<0.01, ***P<0.001 (Student's t-test).

FIG. 5A-C. Ectopic expression of 11βHSD1 in MCF7 inhibits cell proliferation and OCDO reverses this effect. MCF7 cells were transfected by electroporation with a plasmid coding either the enzymes 11βHSD1 (HSD1) or the control empty vector (mock) and analyzed as followed: (A) the expression of 11βHSD1 was confirmed by immunoblotting using specific antibody against 11βHSD1 and normalized with actin; (B) The production of CT was determined by incubating the mock or the HSD1-transfected cells with ¹⁴C-OCDO for 72 h at 37° c. The radioactive metabolites of interest were analyzed as in the legend of FIG. 4B. (C) The proliferation of the mock- or the HSD1-transfected MCF7 cells treated or not with 5 μM OCDO for 24 h were analyzed as in FIG. 2A. The results in B and C are the mean (±s.e.m) of three to five experiments, **P<0.01, ***P<0.001 (Student's t-test), ns: non specific.

FIG. 6A-F. Knock-down of 11βHSD2 decreases OCDO production, cell proliferation, invasion and survival in MCF7 cells. MCF7 cells (5×10⁶ cells) were transfected by electroporation with a plasmid expressing a short-hairpin RNA (shRNA) against 11βHSD2 or a control shRNA, two clones (A and B) were selected and analyzed as followed: (A) the knock down of 11βHSD2 expression in MCF7 was confirmed by immunoblotting as described in FIG. 4 or by qPCR; (B, C) The quantification of cortisone (B) or OCDO (C) produced by the sh-Control (shC A and B) or the shHSD2-transfected cells (shHSD2 A and B) were measured as described in FIG. 4. (D, E) The proliferation of sh-C or shHSD2 was measured using quantification of DNA BrDU incorporation (D) as described in FIG. 2A or by cell counting (E). (F) the formation of colony by sh-C or shHSD2 MCF7 cells was quantified after cell fixing and crystal violet staining. The results are the mean (±s.e.m) of three to five experiments,*P<0.05, **P<0.01 (Student's t-test).

FIG. 7A-F. Knock-down of 11βHSD2 decreases cell proliferation, invasion and survival in MCF7 cells as well as tumor growth and OCDO reverses these effects. shC or shHSD2 MCF7 cells were analyzed as followed: (A) The proliferation of sh-C or shHSD2 cells treated or not with OCDO 5 μM 24 h was measured using quantification of DNA BrDU incorporation as described in FIG. 2A. (B) The proliferation of sh-C or shHSD2 cells treated or not with increasing concentration of cortisone for 24 h was measured as in (A). (C) The invasiveness of sh-C or shHSD2 cells treated or not with OCDO 5 μM for 72 h was assayed using matrigel-coated filters. (D) the formation of colony by sh-C or shHSD2 cells treated or not with OCDO 1 μM was quantified as described in FIG. 6F. (E) Mice were implanted s.c. with shC or shHSD2 MCF7 cells (5×10⁶ cells) and animals (8 per group) were treated s.c. every day starting on the day of implantation with either the solvent vehicle or OCDO (16 μg/kg). Animals were monitored for tumor growth twice a week. The data are representative of three independent experiments. The mean tumor volume±s.e.m is shown, **P<0.01, ***P<0.001 (analysis of variance (ANOVA), Dunnett's post test). (F) Mean (±s.e.m) of Ki67 positive cell number determined from IHC staining of shC or shHSD2 MCF7 tumor sections from (E), n=8, *P<0.05 (Student's t-test) using HistoQuant, Pannoramic Viewer (3DHistech).

### EXAMPLE

Material & Methods

Materials

Chemicals [3H]cortisol, [3H]cortisone and [14C]cholesterol were purchased from Perkin Elmer. The radiochemical purity of the compounds was verified by thin-layer chromatography (TLC) and was greater than 98%. Autoradiography experiments were done with GE Healthcare or Kodak phosphor screens. Fulvestrant (ICI 182780) used in vivo was a generous gift from the Institute Claudius Regaud (France). The NEON Transfection system was from Invitrogen, the BrdU cell proliferation elisa was from Roche Diagnosic, all plasmids were from Origene (HSD1 sc109325, HSD2 sc122552, H6PDH sc117481, DHCR7 sc110871, EBP or D8D7I sc116006. Other compounds and chemicals were from Sigma-Aldrich (St. Louis, Mo.), and solvents from VW. The antibodies were from the following company: 11βHSD2 (Santa cruz, H-145), 11βHSD1 (Abeam, EPR9407(2)), H6PDH (Santa Cruz, C-10), EBP (Abgent, RB23728) and DHCR7 (Abeam, ab170388).

Animals

Female C57BL/6 Charles River Laboratories (France), Balb/c and NMRI Nude mice (6 weeks old) Janvier (France) were maintained in specific pathogen-free conditions and were included in protocols only following 2 weeks quarantine. All of the animal procedures for the care and use of laboratory animals were conducted according to the ethical guidelines of our institution and followed the general regulations governing animal experimentation.

Cell Culture

MCF-7, SKBR3, MDA-MB-231, MDA-MB-468, HEK293T and E0771 cells were from the American Type Culture Collection (ATCC) and cultured until passage 30. TS/A cells were provided by Dr P. L. Lollini (Bologna, Italy) and MELN cells were a generous gift of Dr. G. Freiss (Montpellier, France). MCF-7 cells were grown in RPMI 1640 medium (Lonza) supplemented with 5% fetal bovine serum (FBS) (Dutcher), SKBR3 cells in Mc Coy's medium (invitrogen) 10% SVF, TSA and MDA-MB-468 cells in RPMI 10%, E0771 in RPMI 10% SVF HEPES 10 mM and HEK 293T and MDA-MB-231 in DMEM (Lonza) 10% SVF. All the cells lines were cultured in 1% penicillin and streptomycin (50 U/ml) (invitrogen) in a humidified atmosphere with 5% CO₂ at 37° C.

Cell Transfection

MCF7 or HEK293T cells (5×10⁶ cells) were transfected with 5 μg of the indicated plasmid using the NEON Transfection System and according to the manufacturer's recommendations. Stable clones were established after MCF7 cells were separately transfected with four different shRNA plasmids targeting 11βHSD2 (11βHSD2 shRNA) or with a control shRNA (11βHSD2 SureSilencing ShRNA plasmid, Qiagen). Cells were then cultured for 3 weeks in presence of 0.5 mg/ml puromycin (Life Technologies). Several clones were analyzed by immunoblot analysis and real time RT-qPCR for the knock down of the expression of the protein of interest.

Analysis of Tumours

Exponentially growing MCF7, ShMCF7, E0771, MDA-MB231, MDA-MB468 and TS/A cells were collected, washed twice in PBS and resuspended in PBS. TS/A and E9771 tumours were prepared by subcutaneous transplantation of 35×10³ cells or 3×10⁵ cells respectively in 100 μl PBS into the flank of BALB/c or C57B16 mice. For other tumors, 5 to 10×10⁶ cells in 2004, PBS/matrigel (1/1) were injected into the flank of NMRI nude mice. Animals were treated as indicated in the legends. Animals were examined daily, and body weights were measured twice per week. In all the experiments, the tumor volume was determined by direct measurement with a caliper and was calculated using the formula (width²× length)/2. Tumors were either frozen in liquid nitrogen or fixed in 10% neutral-buffered formalin and embedded in paraffin for immunohistochemical analysis. Paraffin sections were stained with haematoxylin and eosin for histomorphological analyses. Immunohistochemical staining was done on paraffin-embedded tissue sections, using a specific Ki67 antibody (Dako).

Chemical Synthesis

5,6α-EC, 5,6β-EC were synthesized as reported^{10, 20}. CT and OCDO were synthesized as reported²¹.

Metabolic Activity Assay in Intact Cells

Cells were plated on six-well plates (1×10⁵ cells/well) in the appropriate complete medium. One day after seeding, cells were treated with either ¹⁴C-CT (1 μM, 10 μCi/μmol-1 μl/dish) or ¹⁴C-OCDO (1 μM, 10 μCi/μmol-1 μL/dish) or ³H-cortisol (200 nM, 89 Ci/mmol-1 μL/dish) or ³H-cortisone (200 nM, 89 Ci/mmol-1 μL/dish) or ¹⁴C-αEC or ¹⁴C-βEC (600 nM, 20 μCi/μmol-1 μl/dish) at the indicated times. After incubation, cells were washed, scraped, and neutral lipids were extracted with chloroform-methanol as described in¹¹ and then separated by TLC using Ethyl Acetate as eluant for ¹⁴C-CT and ¹⁴C-OCDO or chloroform-methanol (87:13, v/v) for ³H-cortisol or ³H-cortisone adapted from²². The radioactive lipids were detected by autoradiography (KODAK, BioMax MS Film). The positions of the metabolite of interest were determined using purified ¹⁴C or ³H standards and the region corresponding of CT, OCDO, cortisol or cortisone was scraped and quantified using a beta counter.

Cell Proliferation Assay

Cells, MCF7 (4×10³), MCF7-sh11bHSD2 (4×10³), SKBR3 (2.5×10³), TSA (2.5×10³), MDA-MB231 (5×10³) and MDA-MB468 (5×10³), were seeded in 96-well plates and cultured in complete medium for 24 h. Cells were then treated for 24 h with either the indicated concentration of OCDO, cortisol or cortisone or with 1 μM RU486 or ICI182780 added 30 mn before other treatment. At the end of this time, cells were incubated with BrDU for an additional 8 h and then evaluated for proliferation using the ELISA kit, Roche Diagnostic, as indicated by the manufacturer.

Cell Invasion Assay

Invasion assays were carried out using Bio-Coat migration chambers (BD Falcon) with 8 μm filters previously coated with matrigel. Cells, MCF7-sh11βHSD2 or MCF7-shC (1×10³), were plated in the upper chambers in SVF free medium and the chemoattractant (10% FBS) was added in the lower chambers. After incubating cells in absence or presence of OCDO (5 μM) for 72 h at 37° C. in 5% CO2 incubator, cells that had migrated through the filters were fixed (3.7% PFA) and stained (aqueous crystal violet 0.05%). The entire membranes were mounted on glass slides, and were counted under a microscope. All experiments were performed in duplicate.

Clonogenic Assay

Cells, MCF7-sh11bHSD2 (5×10³), MCF7-shC (5×10³) or TSA (3×10³) were seeded in duplicate in 35 cm² diameter dish. Twenty four hours after, cells were treated either with OCDO 1 μM or solvent vehicle and the treatment was repeated every 3 days. At day 10, colonies were fixed with 3.7% PFA, stained with an aqueous crystal violet solution (0.05%) and the number of colonies was counted.

Luciferase Assay

MELN cells expressing luciferase in an estrogen-dependent manner²³ or MCF7 co-transfected as described above with the plasmid coding the human glucocorticoid receptor hGR and a plasmid GREluc were routinely grown in DMEM or RPMI 1640 respectively supplemented with 5% FBS (Dutcher). Experiments were carried out as described previously²³. Briefly, 50×10³ cells per well were seeded in 12-well plates and grown for 4 days in phenol red-free medium, containing 5% dextran-coated charcoal-treated FCS. Then, cells were treated for 16 hours with the indicated compounds. At the end of the treatment, cells were washed with PBS and lysed in 250 μL of lysis buffer (Promega). Luciferase activity was measured using the luciferase assay reagent (Promega), according to the manufacturer's instructions. Protein concentrations were measured using the Bradford technique to normalize the luciferase activity data. For each condition, the mean luciferase activity was calculated from the data of three independent wells.

Immunoblotting

Cells treated or not as indicated were washed with ice-cold PBS, scraped, and centrifuged at 1200 rpm for 5 min at 4° C. The pellets were resuspended in 100 μL of extraction buffer (50 mM Tris pH 7.4; 5 mM NaCl; 1% tritonX100; 10% glycerol) with 1% protease inhibitor cocktail (Sigma Aldrich), vortexed and centrifuged at 10,000×g for 10 min at 4° C. Whole cell extracts were fractionated by SDS PAGE and transferred to a polyvinylidene difluoride membrane using a transfer apparatus according to the manufacturer's protocols (Life Technologies). After incubation with 5% nonfat milk in TBST (10 mM Tris, pH 8.0, 150 mM NaCl, 1% Tween 20) for 60 min, the membrane was incubated with antibodies against 11βHSD2 (1:1000), 11βHSD1 (1:500), H6PDH (1:500), EBP (1:500) and DHCR7 (1:200) or actin (1:10000, Merck Millipore, C4) at 4° C. overnight. Membranes were washed three times for 10 min and incubated with a 1:10000 dilution of horseradish peroxidase conjugated anti-mouse or anti-rabbit antibodies for 1 h. Blots were washed with TBST three times and developed with the ECL system (Amersham Biosciences) according to the manufacturer's protocols.

RNA Isolation and qPCR Analysis

Total RNA from cultured cells were isolated using TRIzol Reagent® (Invitrogen). RNA was quantified using nanodrop (thermofisher). Total RNA (1 μg) was reverse transcribed using iScript cDNA synthesis kit (Bio-Rad) according to the manufacturer's instructions. qRT-PCR was performed with an iCycler iQreal-time PCR detection system (Bio-Rad) using iQ SYBR Green Supermix (Bio-Rad) and the indicated primers The threshold cycle (Ct) values of genes of interest were normalized with the Ct values of Cyclophiline A1.

Primers:

| | forward | reverse |
|---|---|---|
| cycloA1 | GCA-TAC-GGG-TCC-TGG-CAT- | ATG-GTG-ATC-TTC- |
| | CTT-GTC-C (SEQ ID NO: 5) | TTG-CTG-GTC-TTG-C |
| | | (SEQ ID NO: 6) |
| 11βHSD1 | GA-CAGCGA-GGT-CAA-AAG- | GTC-CTC-CCA-TGA- |
| | AAA (SEQ ID NO: 7) | GCT-TTC-CTG |
| | | (SEQ ID NO: 8) |
| 11βHSD2 | CCA-CCG-TAT-TGG-AGT- | CGC-GGC-TAA-TGT- |
| | TGA-ACA (SED ID NO: 9) | CTC-CTG-G |
| | | (SEQ ID NO: 10) |
| EBP | CAC-AGG-GGT-CTT-AGT-CGT- | CCA-GGT-GAA-TGA- |
| (D8D7I) | GAC (SEQ ID NO: 11) | ACC-CAC-ACA |
| | | (SEQ ID NO: 12) |
| DHCR7 | ACT-GGC-GAG-CGT-CAT-CTT- | TCC-TCG-TTA-TAG- |
| | C (SEQ ID NO: 13) | GTG-GAG-TCT-TG |
| | | (SEQ ID NO: 14) |
| H6PDH | GCA-GAG-CAC-AAG-GAT-CAG- | GGC-AGC-TAC-TGT- |
| | TTC (SEQ ID NO: 15) | TGA-TGT-TGC |
| | | (SEQ ID NO: 16) |

Immunohistochemistry.

All samples were collected with the approval of the Institutional Review Board of the Claudius Regaud Institute. Written informed consent was obtained before inclusion in this study. Patients' clinical characteristics and tumour pathological features were obtained from the medical reports and followed the standard procedures in our institution. Immunohistochemistry was performed on formalin-fixed, paraffin embedded sections of the initial tumor biopsies with the following antibodies: DHCR7 1:50, H6PDH 1:100, EBP 1:500, 11β-HSD1 1:50 and 11β-HSD2 1:50. Immunostaining was blindly analyzed by the pathologist (MLT).

Statistical Analyses.

Tumour growth curves in animals were analysed for significance by analysis of variance with Dunnett's multiple comparison tests. In other experiments, significant differences in the quantitative data between the control and the treated group were analysed using the Student's t-test for unpaired variables. In the figures, *, ** and *** refer to P<0.05, P<0.01 and P<0.001, respectively, compared with controls (vehicle) unless otherwise specified. Prism software was used for all the analyses.

Results

OCDO is a Metabolite of CT.

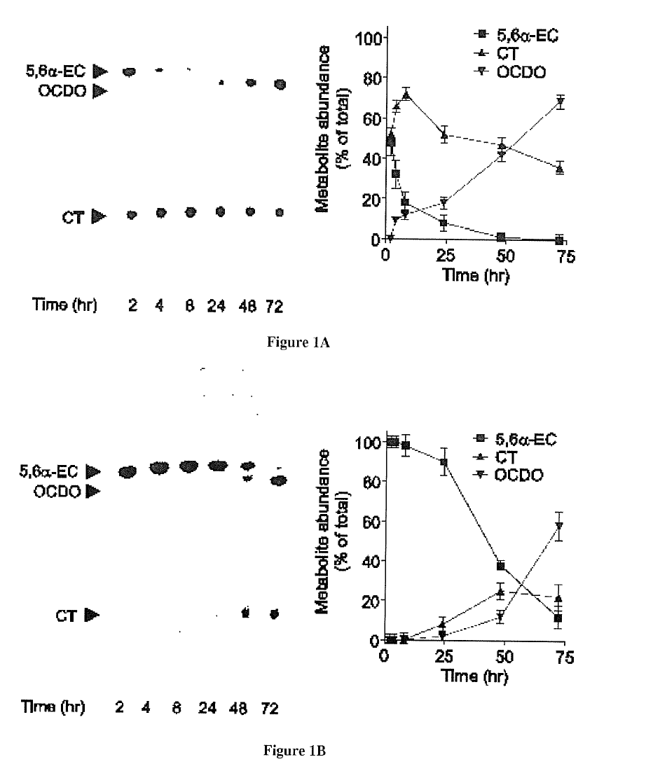

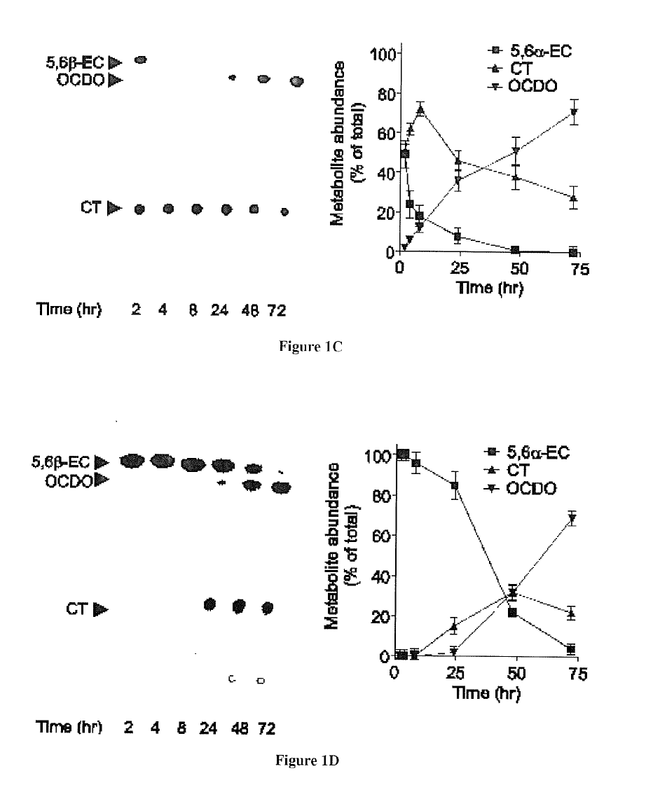

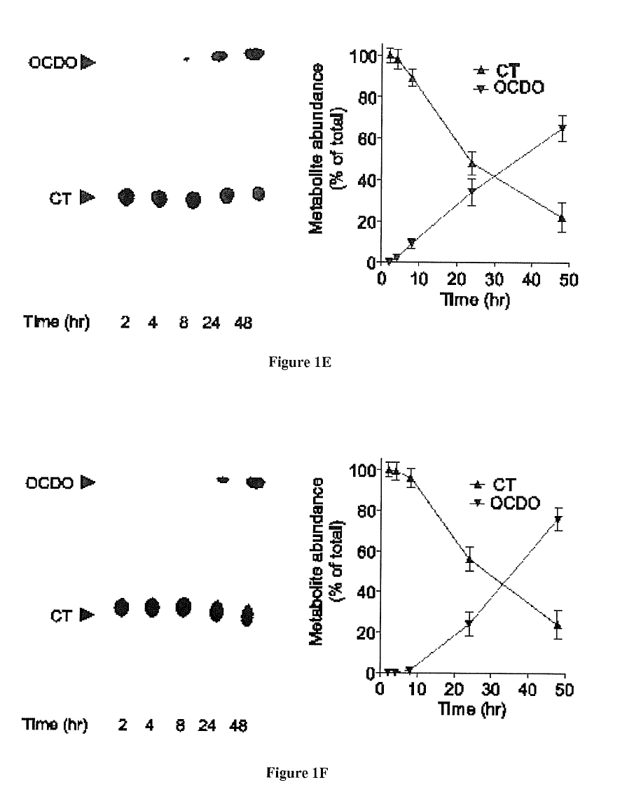

We studied the production of OCDO in breast tumors by incubating MCF7 tumor cells during increasing time with either [¹⁴C]α-EC, [¹⁴C]β-EC or [¹⁴C]-CT. At the indicated time the cells and the media were collected and analyzed separately. As shown in the TLC autoradiograms of FIGS. 1a and 1c, α-EC and β-EC were converted to CT as a result of ChEH activity however, with prolonged incubation times, OCDO production was observed. The formation of OCDO continued when α-EC or β-EC was totally metabolized to CT at 72 h (FIGS. 1a and 1c), indicating that OCDO is formed from CT. Similar experiment performed with [¹⁴C]-CT confirmed that OCDO is a metabolite of CT (FIG. 1e).

OCDO Stimulates Tumor Cell Proliferation and Invasion.

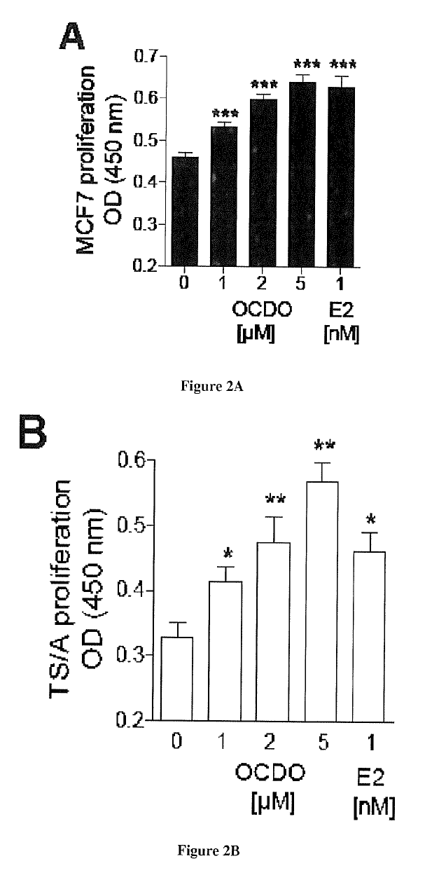

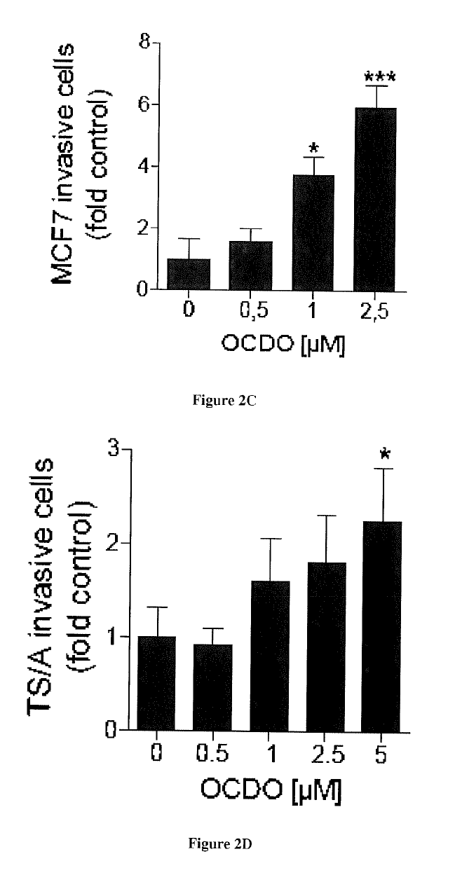

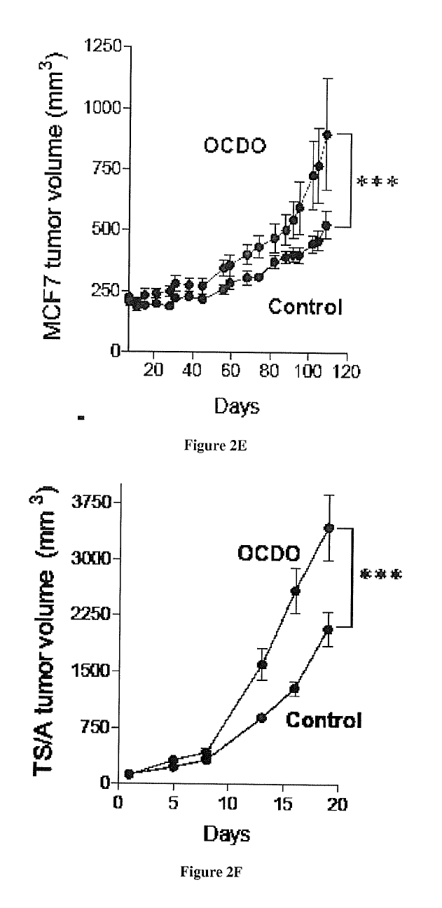

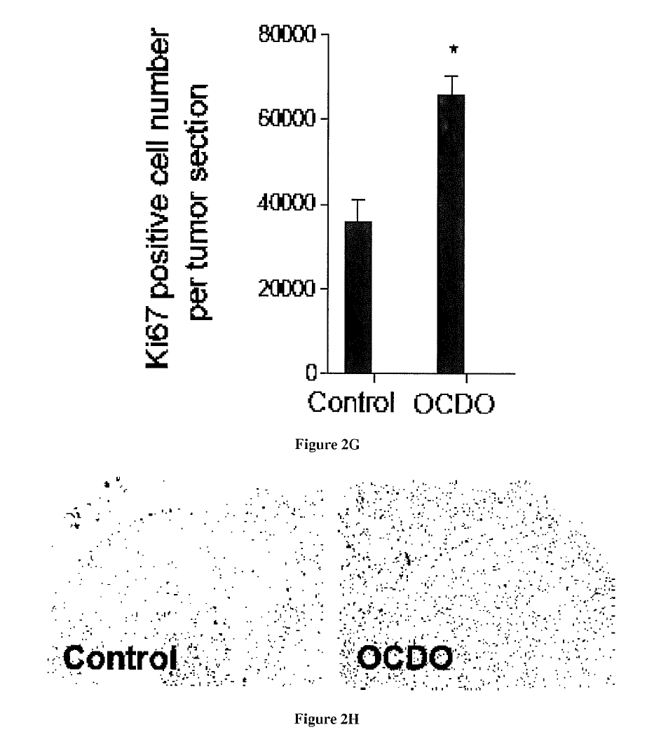

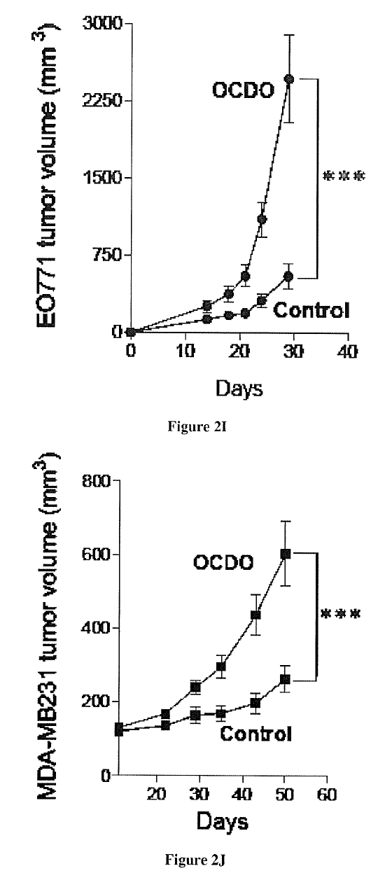

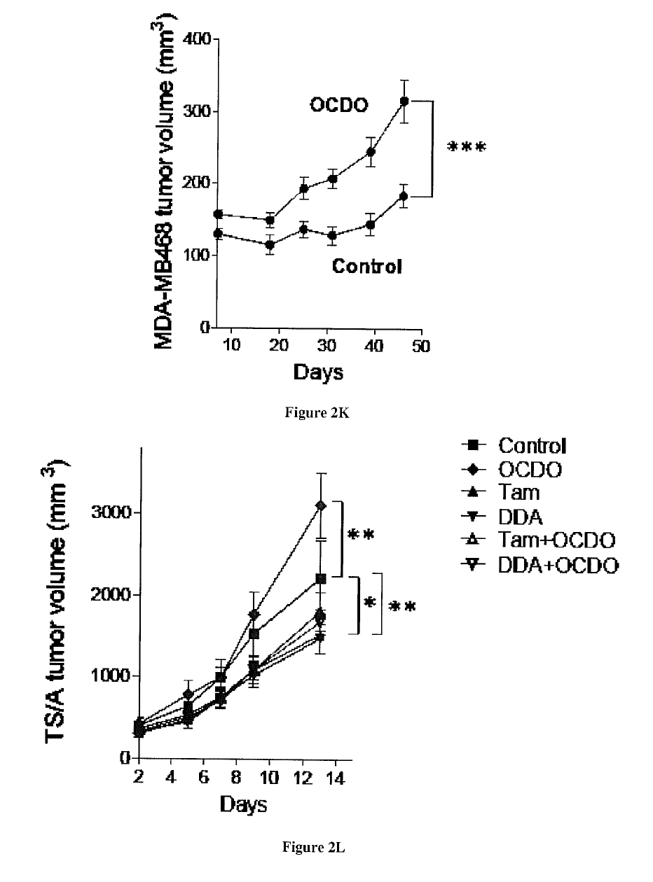

We studied the effects of OCDO on breast tumor cell proliferation and invasion. As shown in FIGS. 2A and 2B, the growth rate of human MCF7 and mouse TS/A cells treated with OCDO for 24 h was increased in a concentration-dependent manner and reached respectively 1.3-fold and 1.7-fold the control. This increased in proliferation was in the same range than with 1 nM estradiol (E2). The invasiveness of MCF7 and TS/A cells treated with OCDO were also increased in a concentration-dependent manner and reached respectively 6-fold and 2.3-fold respectively compared with the control (FIGS. 2C and 2D).

OCDO Stimulates the Proliferation of Breast Tumors Implanted into Mice.

We then assayed whether OCDO stimulates the growth of mammary tumors implanted into mice. OCDO treatment significantly increased the growth of human MCF7 (FIG. 2E) and murine TS/A tumors grafted into immunodeficient or immunocompetent mice respectively compared with the control group (FIGS. 2E and 2F). Histological analysis of MCF7 or TS/A tumors indicated that the proliferative marker Ki67 was increased in OCDO-treated tumors compared with control-treated tumors in both tumor models (FIGS. 2G and 2H). In addition, OCDO stimulates the growth of other tumor models expressing or not the estrogen receptor such as the mouse E0771 and the human MDA-MB231 and MDA-MB468 cells (FIGS. 2I, 2J and 2K respectively).

OCDO Reverses the Tumor Growth Inhibition Effect of ChEH Inhibitors in Mice.

We then assayed the anti-growth effect of Tam or DDA against TS/A tumors in the absence and presence of OCDO. As above, TS/A tumors implanted into immunocompetent mice were treated s.c. every day either with either the solvent vehicle (control), OCDO (50 μg/kg), Tam (56 mg/kg), DDA (20 mg/kg) or the combination of Tam (56 mg/kg)+OCDO (50 μg/kg) or DDA (20 mg/kg)+OCDO (50 μg/kg). As shown in FIG. 2L, after 13 days of treatment, OCDO enhanced TS/A tumor growth by 140% compared with that of the control group (p<0.01). Treatment with Tam or DDA alone significantly inhibited the growth of tumors by 31% (p<0.05) and 33% (p<0.01) respectively compared with the control group. When animals were treated with OCDO and Tam, or OCDO with DDA, the growth of tumors was not statistically different from that of the control group, indicating that the growth inhibitory action of Tam or DDA was reversed by OCDO. These data indicate that the inhibition of OCDO production contributes to the anti-tumor effects of both Tam and DDA.

Identification of the Enzymes that Regulate the Production of OCDO from CT.

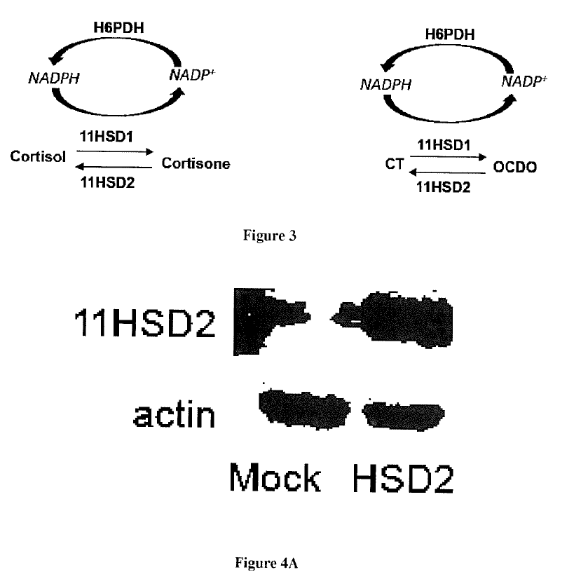

Since the data we obtained argued for the existence of an enzyme distinct from ChEH that metabolizes CT into OCDO, we hypothesized that a hydroxysteroid dehydrogenases (HSD) would catalyze the dehydrogenation (or oxidation) of the alcohol function in position 6 of CT into a ketone in OCDO. Three main classes of HSD has been described (3β-, 17β- and 11β-hydroxy steroid dehydrogenase). A symmetry axis on the steroid backbone makes equivalent the positions 11β and 7α¹⁵, which suggest us that 11βHSD could be a good candidate for this reaction. 11β-HSD exist as two enzymes, 11β-HSD type 2 (11HSD2) which catalyzes the dehydrogenation of cortisol into cortisone and 11β-HSD type 1 (11HSD1) which realizes the reverse reaction and catalyzes the hydrogenation of cortisone into cortisol^{13, 14, 16}(FIG. 3A). Interestingly 11βHSD1 accepts also as substrate 7-ketocholesterol which is transformed into 7-hydroxycholesterol¹⁶. Importantly, 11βHSD2 is expressed in MCF7 while 11βHSD1 is not detected¹⁷, suggesting a possible deregulation of the equilibrium between 11βHSD1 and 11βHSD2 expression in tumor cells, that would favor OCDO production. In accordance with this hypothesis, we characterized significant levels of 11βHSD2 at the mRNA and protein level in various human BC cell lines reflecting different BC subtypes while 11βHSD1 expression was not detectable either at the mRNA or protein levels and all the cell lines tested produced OCDO (Table 1).

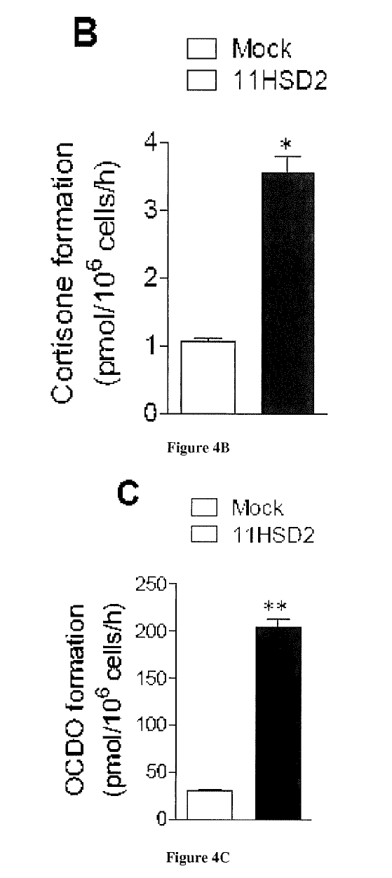

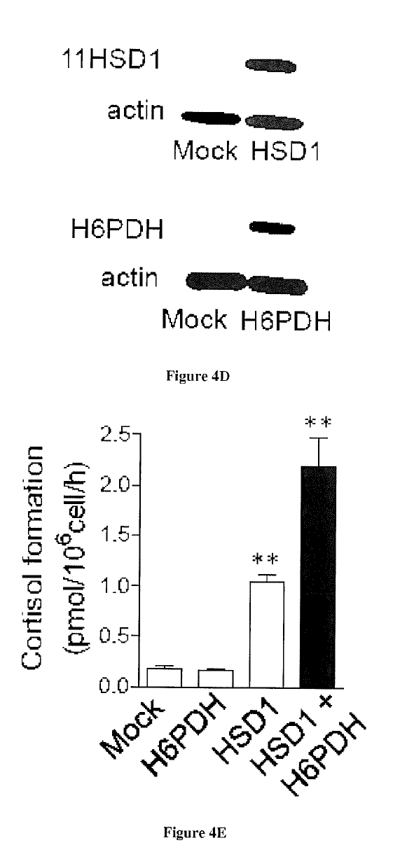

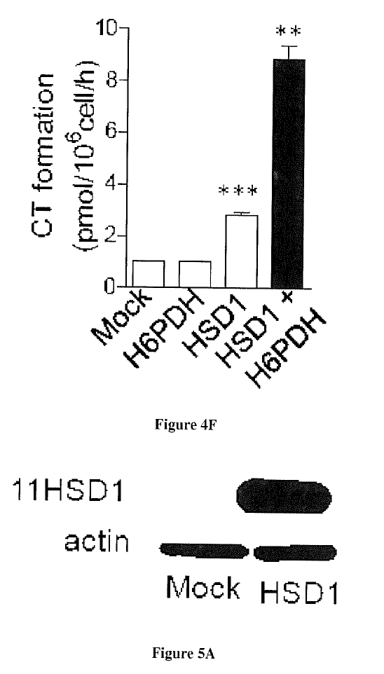

To confirm the implication of 11βHSD2 in the production of OCDO from CT, we transfected HEK-273 cells, a cell model previously used to study cortisol/cortisone metabolism¹⁸, with a plasmid coding either the 11βHSD2 (HSD2) or the empty vector (mock). Immunoblot analysis of mock transfected HEK-273 cells did not detect endogenous 11βHSD2 (FIG. 4A). In contrast, in 11βHSD2-transfected HEK-273 cells, 11βHSD2 was well detected migrating (FIG. 4A). We first measured the capacity of the 11βHSD2-transfected HEK-273 cells to produce cortisone when incubated 8 h with ³H-cortisol. As observed in FIG. 4B, 11βHSD2-transfected HEK-273 cells produced 3-fold more cortisone (3.3 pmol/10⁶ cells/h) than mock-transfected cells (1.1 pmol/10⁶ cells/h), indicating that the encoded enzyme was functional. We then measured the production of OCDO after incubating transfected-HEK-273 cells with [¹⁴C]α-CT for 8 h. As shown in FIG. 4C, 11βHSD2-transfected HEK-273 cells induced a 7-fold increase production of OCDO (195 pmol/10⁶ cells/h) compared with mock-transfected HEK-273 cells (29 pmol/10⁶ cells/h). Together these data indicate that 11βHSD2 is able to produce significant levels of OCDO in addition to cortisone.

To study the implication of 11βHSD1 in the transformation of OCDO into CT, HEK293 cells were transfected with a plasmid coding the 11βHSD1 (HSD1) or the empty vector (mock) and with or without a plasmid coding the H6PDH, the enzyme that produces the cofactor NADPH necessary for 11βHSD1 reductase activity as reported in¹⁸ (FIG. 3A). No endogenous expression of 11βHSD1 or H6PDH was detected in HEK293 cells transfected with the empty vector (mock) by western blot analysis (FIG. 4D). In contrast, in 11βHSD1 and H6PDH transfected-HEK293 cells, the proteins were well detected (FIG. 4D). We then measured the capacity of the HEK293 transfected cells to produce cortisol after incubating with ³H-cortisone. As shown in FIG. 4E, low production of cortisol was measured in the mock-transfected cells or in H6PDH-transfected cells (about 0.20 pmol/10⁶ cells/h). In contrast, 11βHSD1-transfected cells produced 5-fold more cortisol than mock-transfected cells (1.1 pmol/10⁶ cells/h), and this production was increased twice by co-transfecting 11HβSD1 and H6PDH (2 pmol/10⁶ cells/h). Together the data indicated that the transfected enzymes 11βHSD1 and H6PDH are functional. We then measured the production of CT after incubating transfected HEK293 cells with [¹⁴C]-OCDO for 24 h. As shown in FIG. 4F, the production of CT was of about 1 pmol/10⁶ cells/h in cells transfected with the empty plasmid or with H6PDH while the transfection of the plasmid coding 11βHSD1 induced a 3-fold increased production of CT and the co-transfection of H6PDH and 11βHSD1 further increased CT production that reached 8-fold (8.5 pmol/10⁶ cells/h) the levels of the mock-transfected cells. These data indicate that 11βHSD1 is able to produce significant levels of CT in addition to cortisol.

Ectopic Expression of 11βHSD1 in MCF-7 Cells Induces CT Production and Decreases Cell Proliferation and OCDO Treatment Reverses this Effect.

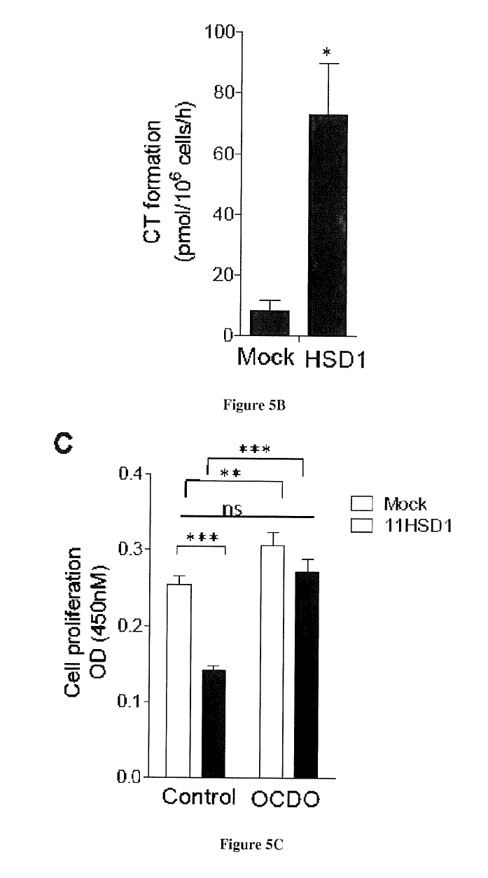

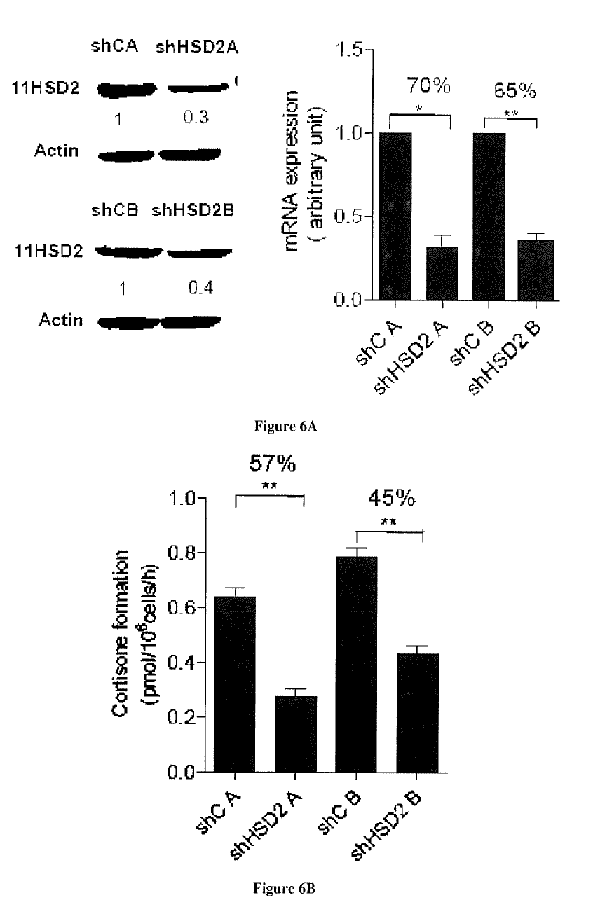

Since MCF7 cells do not express 11βHSD1, we transfected these cells with a plasmid expressing this enzyme (FIG. 5A) and evaluated the impact of its expression on CT production and cell proliferation. As shown in FIG. 5B, the expression of 11βHSD1 in MCF7 cells significantly stimulated OCDO to CT conversion compared with the control (73±12 against 8.5±2.5 pmol/10⁶ cells/h). In addition, the expression of 11βHSD1 in MCF7 cells significantly decreased cell proliferation by 45% and OCDO treatment reversed this effect (FIG. 5C), indicating that 11βHSD1 inhibits cell proliferation through transformation of OCDO into CT.

Knock-Down of 11βHSD2 Decreases Cell Proliferation, Invasion and Survival in MCF7 Cells as Well as Tumor Growth and OCDO Reverses these Effects.

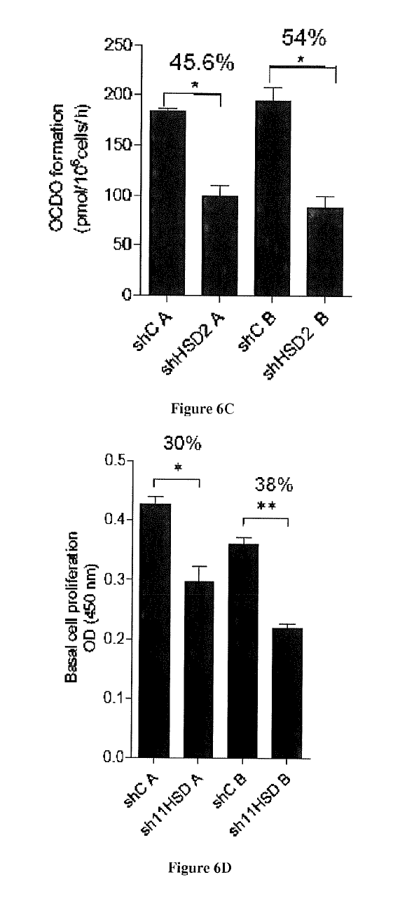

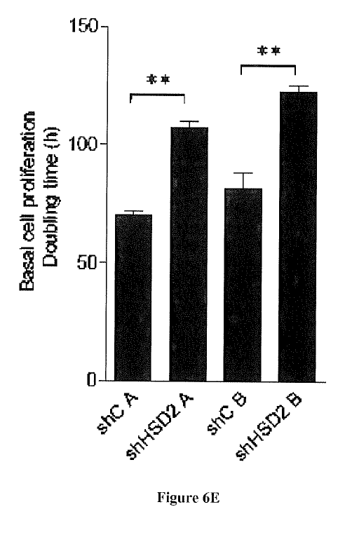

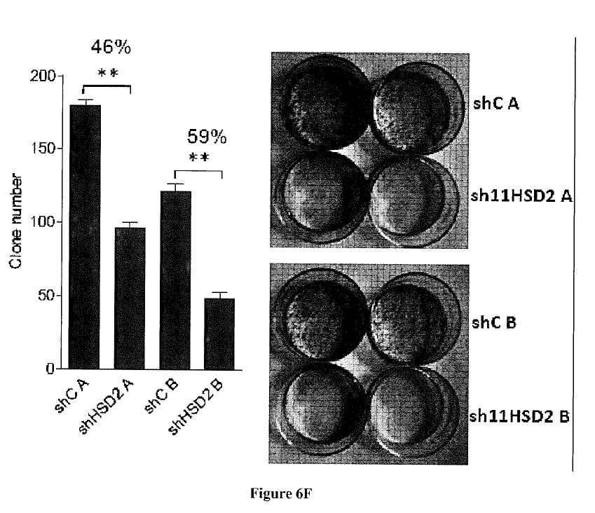

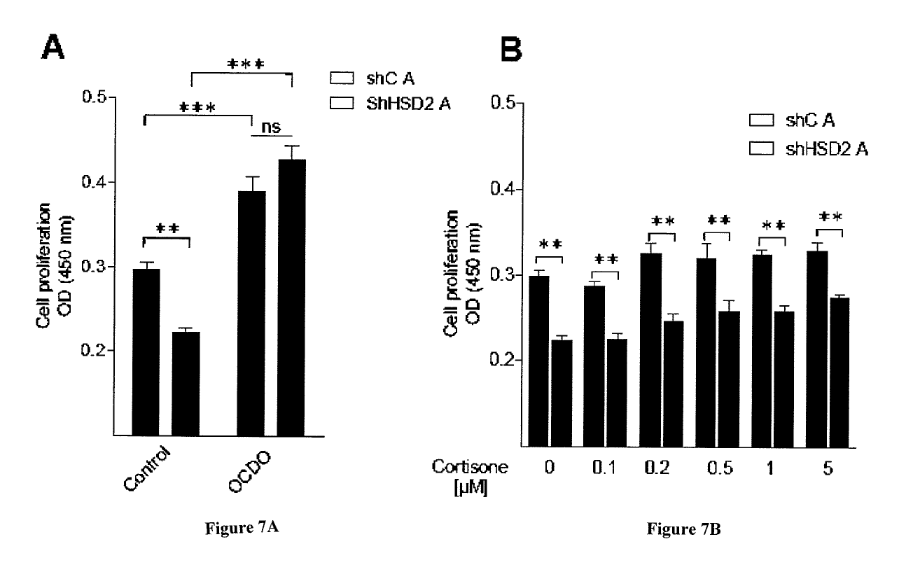

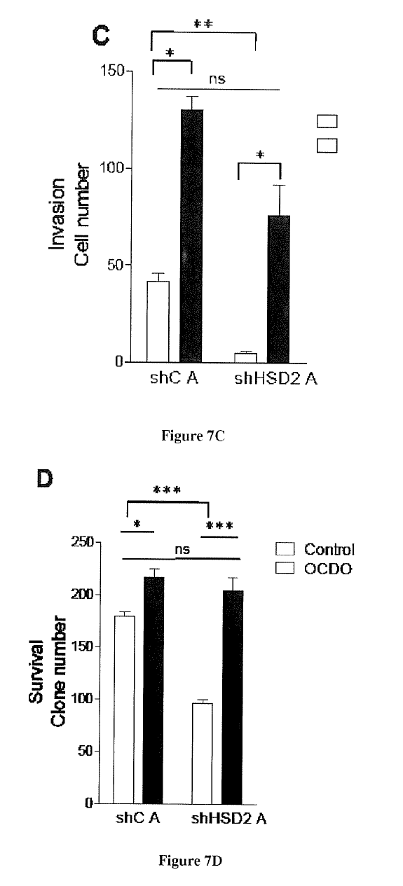

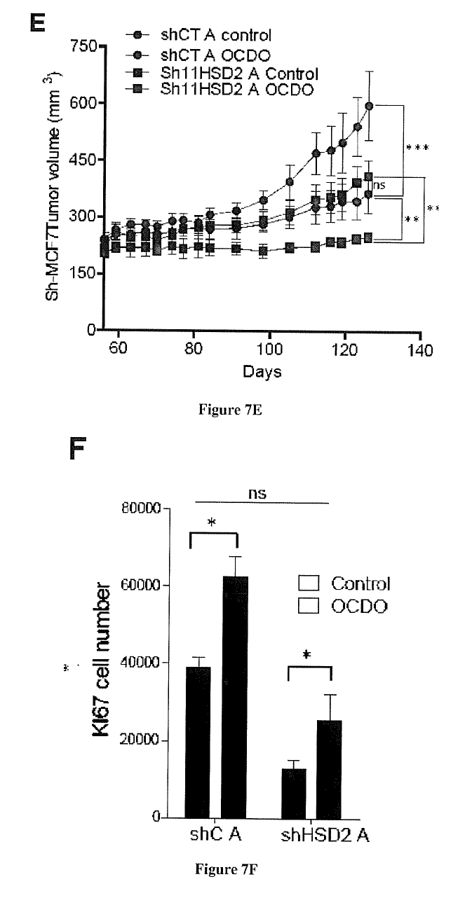

To study the implication of 11βHSD2 in cell proliferation and survival, we knocked down the expression of 11βHSD2 in MCF7 cells by using shRNA against the enzyme or control shRNA. Two stable clones were selected in which the expression of 11βHSD2 was significantly decreased at both protein and mRNA level (sh11HSD2 A and sh11HSD2 B) and compared with shRNA control clones (shC A and shC B) (FIG. 6A). A significant decrease in cortisone and OCDO production was measured in sh11HSD2 A and B clones compared with shC A and B control clones (FIGS. 6B and 6C respectively). Basal cell proliferation of the two sh11HSD2 clones was significantly decreased (FIG. 6D) and their doubling time was increased by 142% and 150% (FIG. 6E) compared with control clones. Moreover, the knock-down of 11βHSD2 expression also significantly decreased cell survival in a clonogenic assay (FIG. 6F). Importantly, we determined that OCDO was able to reverse the inhibition of cell proliferation induced by decreasing the expression of 11βHSD2 in sh11HSD2 (FIG. 7A) while cortisone even at high concentrations did not (FIG. 7B). Similarly, OCDO reversed the inhibition of cell invasion (FIG. 7C) and cell survival (FIG. 7D) mediated by the knock-down of 11βHSD2. Together these results indicate that 11βHSD2 controls cell proliferation, survival and cell invasion through OCDO production. We then tested the impact of 11βHSD2 knock-down in vivo on ShC or sh11HSD2 cells xenografted in immunodeficient mice. As shown in FIG. 7E, the basal growth of sh11HSD2 tumors was significantly decreased (by 29%) compared with that of shC tumors. Importantly, subcutaneous treatment with OCDO (15 μg/kg, 5 days/week) reversed the growth inhibition of sh11HSD2 tumors to a level similar to the growth of shC tumors. KI67 staining of the tumors indicated that cell proliferation was increased in ShC tumors through OCDO treatment and decreased in sh11HSD2 tumors, and OCDO reversed the growth inhibition of sh11HSD2 tumors. Together, these date indicate that 11βHSD2 controls tumor growth through OCDO production.

Expression of the Enzymes Regulating OCDO Production in Breast Cancer Samples and Normal Matched Tissue.

We then explored the expression of the enzymes regulating OCDO in breast patient samples and normal adjacent tissues. As shown in Table 2, immunohistology analyses showed that 11βHSD2 was mainly expressed in breast tumors (93% of 49 samples) and weakly or not in normal adjacent tissues (8% of 46 samples). 11βHSD2 was also observed in the blood vessels in 43% of breast tumor samples. 11βHSD1 was poorly present either in the tumor samples (25% of 48 samples) and in the normal tissue (38% of 42 samples) and H6PDH showed the same tendency (34% of 32 tumor samples and 57% of the 42 normal cases), however the expression of both enzymes was lower in tumors compared to normal tissue. DHCR7 and D8D7I were found expressed both in tumor and normal tissues. However, for DHCR7 a strong expression was observed in 54% of the 49 tumor samples compared with normal tissue and interestingly the expression of the enzyme was increased in the adipocytes surrounding the tumors (78% of the samples) compared with the adipocytes that were distant. For D8D7I, a strong staining was also observed in 63% of the 50 tumor samples compared with the normal tissues. Together these results indicate that the expressions of the enzymes producing OCDO are increased or high in tumors compared with normal tissue.

### DISCUSSION

The present study identifies new functions for 11-βHSD2 and 11-βHSD1 as being the enzymes involved in the inter-conversion of OCDO and CT. Thus, several enzymes are involved in the production and regulation of OCDO production. Previously, we showed that the ChEH, that is carried out by D8D7I and DHCR7, mediates the transformation of 5,6-EC into CT that leads to the production of OCDO in tumors^{8, 10}. The inhibition of ChEH by molecules such Tam or DDA blocks the production of OCDO and its proliferative effect in cancer cells and tumors, while the addition of OCDO reverses these effects^{8, 10} and present study. Here, we show that 11-βHSD2 and 11-βHSD1, which are known to regulate the metabolism of the glucocorticoids, cortisol and cortisone in human, are involved in the next step to produce OCDO from CT or to produce CT from OCDO respectively. Importantly, 11-βHSD2 controls both in vitro and in vivo tumor cell proliferation through OCDO production, in add back experiments in which 11-βHSD2 expression has been attenuated. Conversely, 11-βHSD1 re-expression in tumor cells lacking this enzyme inhibits cell proliferation through transformation of OCDO into CT and OCDO addition reverses this effect. Thus, activation of 11-βHSD2 not only promotes inflammation and decreases the inhibition of cell proliferation induced by the inactivation of cortisol into cortisone but also produces an onco-metabolite OCDO that actively participates to cancer proliferation and invasion. Importantly, OCDO increases the proliferation of estrogen-positive or estrogen-negative breast tumors, indicating that OCDO may contribute to stimulate tumor progression even in the absence of estrogens. The 11-βHSD2 enzyme is exclusively oxidative, converting the active cortisol to the inactive cortisone and requiring NAD as cofactor. 11-βHSD1 presents a dual reductase and dehydrogenase activity, depending for the dehydrogenase activity of the presence of H6PDH that produces the co-factor NADP¹⁸. In absence of H6PDH expression, 11-βHSD1 will work as a dehydrogenase as reported in human omental preadipocytes¹⁹. According to our results, the absence or the decrease level of 11-βHSD1 in tissues expressing 11-βHSD2 would favour the production of OCDO in addition to converting cortisol to cortisone. Similarly, the decrease or the absence of H6PDH may favour the dehydrogenase activity of 11-βHSD1 and thus the production of OCDO and cortisone. In the present study, the immunohistology analyses indicate that the expressions of the enzymes producing OCDO, 11βHSD2, D8D7I and DHCR7, are increased or high in tumors compared with normal tissues and that the enzymatic equilibrium between 11βHSD2 and 11βHSD1/H6PDH is shifted toward the production of OCDO in tumors. These results are consistent with the pro-tumor and pro-invasive activity of OCDO that we report in the present study and its secretion by the tumor cells should contribute to tumor proliferation and aggressiveness. 11βHSD2 is also present in cells of the vasculature in 43% of the tumor samples, indicating that OCDO may be secreted in the blood fluid to act at distance of the tumor in addition to an autocrine action and it may actively participate to tumor invasion. An effect of OCDO on the proliferation of blood vessels could be also considered.

Thus, the discovery of OCDO and its pro-tumor effect as well as the discovery of the enzymes regulating its production are important findings that should have major implications in tumor biology and therapy. Therefore, the activation of OCDO production as well as the expression of the enzymes producing or regulating OCDO could be markers of cancer and of the efficacy of anti-cancer compounds such as Tam or DDA.

TABLE 1
Expression and activity of 11βHSD1 and 11βHSD2 in BC
tumor cells. Different subtypes of breast cancer cells were
analyzed for the expression of 11βHSD1 and 11βHSD2 by
either qPCR or immunobloting as well as OCDO production
by incubating tumor cells with ¹⁴C-αEC for 24 h as described
in FIG. 1. The amount of OCDO formed per hour was
normalized to the number of cells. The results are the mean
(±s.e.m) of two to three experiments.

| | | | 11HSD2 | | |
|---|---|---|---|---|---|
| | | | | | OCDO production |
| | 11HSD1 | | | | pmol/10⁶ |
| Cells | mRNA | protein | mRNA | protein | cells/h ± s.e.m |
| MCF-7 | >35 | − | 26.5 | + | 10.6 ± 2 |
| BT474 | >35 | − | 26 | + | 5.76 ± 1.5 |
| SKBr3 | >35 | − | 27.1 | + | 3.5 ± 0.3 |
| ZR751 | >35 | − | 25.2 | + | 9.3 ± 1 |
| MDA-MB- | >35 | − | 24.2 | + | 23.9 ± 3.4 |
| 468 | | | | | |
| MDA231- | 28.2 | − | 28.5 | + | 2 ± 0.4 |
| MB- | | | | | |
| HCC1937 | 33 | − | 28.0 | + | 7.3 ± 0.3 |
| LCC1 | >35 | − | 23.7 | + | 19.5 ± 2.2 |
| LCC2 | >35 | − | 23.6 | + | 29.3 ± 9 |
| (TamR) | | | | | |
| RTx6 | >35 | − | 25.4 | + | 2 ± 0.1 |
| (TamR) | | | | | |
| TS/A | 35 | − | 26 | ND | 9.5 ± 0.1 |
| E0771 | 35 | − | 24 | ND | 22.5 ± 4 |

ND: not deternnined
TamR: cells derived from MCF7 resistante to tamoxifen

TABLE 2
Expression of enzymes regulating OCDO production in breast
tumor patient samples and normal matched tissues. Immunohistology
analyses using specific antibodies against the enzymes
regulating OCDO production were scored as described in
the “Materials and Methods” section.

| | |
|---|---|
| Cancer | Adjacent normal tissue |

| | n | % | n | % |
|---|---|---|---|---|
| 11HSD2 | 49 | 93 | 46 | 8 |
| 11HSD1 | 48 | 25 | 42 | 38 |
| H6PDH | 32 | 34 | 42 | 57 |
| DHCR7 | 49 | 83 | 43 | 74 |
| | | 54* | | |
| D8D7I | 50 | 98 | 43 | 70 |
| | | 63* | | |

*High expression compared with normal tissue

### REFERENCES

Throughout this application, various references describe the state of the art to which this invention pertains. The disclosures of these references are hereby incorporated by reference into the present disclosure.
1. Jordan, V. C. Chemoprevention of breast cancer with selective oestrogen-receptor modulators. Nat Rev Cancer 7, 46-53 (2007).
2. Pitroda, S. P., Khodarev, N. N., Beckett, M. A., Kufe, D. W. & Weichselbaum, R. R. MUC1-induced alterations in a lipid metabolic gene network predict response of human breast cancers to tamoxifen treatment. Proc Natl Acad Sci USA 106, 5837-5841 (2009).
3. Silvente-Poirot, S. & Poirot, M. Cholesterol metabolism and cancer: the good, the bad and the ugly. Curr Opin Pharmacol 12, 673-676 (2012).
4. Poirot, M., Silvente-Poirot, S. & Weichselbaum, R. R. Cholesterol metabolism and resistance to tamoxifen. Curr Opin Pharmacol 12, 683-689 (2012).
5. Nelson, E. R. et al. 27-Hydroxycholesterol links hypercholesterolemia and breast cancer pathophysiology. Science 342, 1094-1098 (2013).
6. Silvente-Poirot, S. & Poirot, M. Cancer. Cholesterol and cancer, in the balance. Science 343, 1445-1446 (2014).
7. de Medina, P. et al. Dendrogenin A arises from cholesterol and histamine metabolism and shows cell differentiation and anti-tumour properties. Nat Commun 4, 1840 (2013).
8. de Medina, P., Silvente-Poirot, S. & Poirot, M. Methods for determining the oncogenic condition of cell, use thereof, and methods for treating cancer. Word Patent, 2010/149941 (2010).
9. Sevanian, A. & McLeod, L. L. Catalytic properties and inhibition of hepatic cholesterol-epoxide hydrolase. J Biol Chem 261, 54-59. (1986).
10. de Medina, P., Paillasse, M. R., Segala, G., Poirot, M. & Silvente-Poirot, S. Identification and pharmacological characterization of cholesterol-5,6-epoxide hydrolase as a target for tamoxifen and AEBS ligands. Proc Natl Acad Sci USA 107, 13520-13525 (2010).
11. Segala, G. et al. 5,6-Epoxy-cholesterols contribute to the anticancer pharmacology of Tamoxifen in breast cancer cells. Biochem Pharmacol 86, 175-189 (2013).
12. Sola, B. et al. Antiestrogen-binding site ligands induce autophagy in myeloma cells that proceeds through alteration of cholesterol metabolism. Oncotarget 4, 911-922 (2013).
13. Chapman, K., Holmes, M. & Seckl, J. 11beta-hydroxysteroid dehydrogenases: intracellular gate-keepers of tissue glucocorticoid action. Physiol Rev 93, 1139-1206 (2013).
14. Gathercole, L. L. et al. 11beta-Hydroxysteroid dehydrogenase 1: translational and therapeutic aspects. Endocr Rev 34, 525-555 (2013).
15. Lathe, R. & Kotelevtsev, Y. Steroid signaling: ligand-binding promiscuity, molecular symmetry, and the need for gating. Steroids 82, 14-22 (2014).
16. Odermatt, A. & Nashev, L. G. The glucocorticoid-activating enzyme 11 beta-hydroxysteroid dehydrogenase type 1 has broad substrate specificity: Physiological and toxicological considerations. J Steroid Biochem Mol Biol 119, 1-13 (2010).
17. Kim, C. H. & Cho, Y. S. Selection and optimization of MCF-7 cell line for screening selective inhibitors of 11beta-hydroxysteroid dehydrogenase 2. Cell Biochem Funct 28, 440-447 (2010).
18. Atanasov, A. G., Nashev, L. G., Schweizer, R. A., Frick, C. & Odermatt, A. Hexose-6-phosphate dehydrogenase determines the reaction direction of 11beta-hydroxysteroid dehydrogenase type 1 as an oxoreductase. FEBS Lett 571, 129-133 (2004).
19. Bujalska, I. J., Walker, E. A., Hewison, M. & Stewart, P. M. A switch in dehydrogenase to reductase activity of 11 beta-hydroxysteroid dehydrogenase type 1 upon differentiation of human omental adipose stromal cells. J Clin Endocrinol Metab 87, 1205-1210 (2002).
20. de Medina, P., Paillasse, M. R., Payre, B., Silvente-Poirot, S. & Poirot, M. Synthesis of new alkylaminooxysterols with potent cell differentiating activities: identification of leads for the treatment of cancer and neurodegenerative diseases. J Med Chem 52, 7765-7777 (2009).
21. Voisin, M., Silvente-Poirot, S. & Poirot, M. One step synthesis of 6-oxo-cholestan-3beta,5alpha-diol. Biochem Biophys Res Commun 446, 782-785 (2014).
22. Alikhani-Koupaei, R. et al. Identification of polymorphisms in the human 11beta-hydroxysteroid dehydrogenase type 2 gene promoter: functional characterization and relevance for salt sensitivity. FASEB J 21, 3618-3628 (2007).
23. de Medina, P. et al. The prototypical inhibitor of cholesterol esterification, Sah 58-035 [3-[decyldimethylsilyl]-n-[2-(4-methylphenyl)-1-phenylethyl]propanamide], is an agonist of estrogen receptors. J Pharmacol Exp Ther 319, 139-149 (2006).

1. A method of diagnosing cancer in a subject comprising the steps of i) determining the expression level of 11βHSD1 and/or 11βHSD2 in a sample obtained from the subject, ii) comparing the expression level determined at step i) with its predetermined reference value and ii) concluding that the subject suffers from a cancer when the expression level of 11βHSD1 is lower than its predetermined reference value or when the expression level of 11βHSD2 is higher than its predetermined reference value.

2. A method for determining the survival time of subject suffering from a cancer comprising the steps of i) determining the expression level of 11βHSD1 and/or 11βHSD2 in a tumor sample obtained from the subject, ii) comparing the expression level determined at step i) with its predetermined reference value and ii) concluding that the subject will have a long survival time when the expression level of 11βHSD1 is higher than its predetermined reference value or concluding that the subject will have a short survival time when the expression level of 11βHSD2 is lower than its predetermined reference value.

3. A method for determining whether a subject suffering from a cancer will achieve a response with tamoxifen or dendrogenin A comprising the steps of i) determining the expression level of 11βHSD1 and/or 11βHSD2 in a tumor sample obtained from the subject, ii) comparing the expression level determined at step i) with its predetermined reference value and ii) concluding that the subject will achieve a response with tamoxifen or dendrogenin A when the expression level of 11βHSD1 is higher than its predetermined reference value or when the expression level of 11βHSD2 is lower than its predetermined reference value.

4. The method of claim 1 wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

5. The method of claim 4 wherein the cancer is breast cancer.

6. The method of claim 3 wherein when it is determined that the subject will achieve a response with tamoxifen or dendrogenin A, the method includes a step of administering one or both of tamoxifen and dendrogenin A to the subject.

7. The method of claim 1 wherein when it is determined that the subject suffers from cancer, the method includes a step of administering to the subject at least one of a 11β-HSD2 inhibitor, an inhibitor of 11β-HSD2 expression and a nucleic acid encoding 11β-HSD1.

8. The method of claim 2 wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

9. The method of claim 3 wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

10. The method of claim 8 wherein the cancer is breast cancer.

11. The method of claim 9 wherein the cancer is breast cancer.


SEQUENCE LISTING (16 sequences)

SEQ ID NO: 1 - protein, 292 aa, Homo sapiens
MAFMKKYLLPILGLFMAYYYYSANEEFRPEMLQGKKVIVTGASKGIGREMAYHLAKMGAHVVVTARSKETLQKVVSHCLELGAASAHYIAGTMEDMTFAEQFVAQAGKLMGGLDMLILNHITNTSLNLFHDDIHHVRKSMEVNFLSYVVLTVAALPMLKQSNGSIVVVSSLAGKVAYPMVAAYSASKFALDGFFSSIRKEYSVSRVNVSITLCVLGLIDTETAMKAVSGIVHMQAAPKEECALEIIKGGALRQEEVYYDSSLWTTLLIRNPCRKILEFLYSTSYNMDRFINK

SEQ ID NO: 2 - protein, 405 aa, Homo sapiens
MERWPWPSGGAWLLVAARALLQLLRSDLRLGRPLLAALALLAALDWLCQRLLPPPAALAVLAAAGWIALSRLARPQRLPVATRAVLITGCDSGFGKETAKKLDSMGFTVLATVLELNSPGAIELRTCCSPRLRLLQMDLTKPGDISRVLEFTKAHTTSTGLWGLVNNAGHNEVVADAELSPVATFRSCMEVNFFGALELTKGLLPLLRSSRGRIVTVGSPAGDMPYPCLGAYGTSKAAVALLMDTFSCELLPWGVKVSIIQPGCFKTESVRNVGQWEKRKQLLLANLPQELLQAYGKDYIEHLHGQFLHSLRLAMSDLTPVVDAITDALLAARPRRRYYPGQGLGLMYFIHYYLPEGLRRRFLQAFFISHCLPRALQPGQPGTTPPQDAAQDPNLSPGPSPAVAR

SEQ ID NO: 3 - DNA, 1477 bp, Homo sapiens
gggaaattggctagcactgcctgagactactccagcctcccccgtccctgatgtcacaattcagaggctgctgcctgcttaggaggttgtagaaagctctgtaggttctctctgtgtgtcctacaggagtcttcaggccagctccctgtcggatggcttttatgaaaaaatatctcctccccattctggggctcttcatggcctactactactattctgcaaacgaggaattcagaccagagatgctccaaggaaagaaagtgattgtcacaggggccagcaaagggatcggaagagagatggcttatcatctggcgaagatgggagcccatgtggtggtgacagcgaggtcaaaagaaactctacagaaggtggtatcccactgcctggagcttggagcagcctcagcacactacattgctggcaccatggaagacatgaccttcgcagagcaatttgttgcccaagcaggaaagctcatgggaggactagacatgctcattctcaaccacatcaccaacacttctttgaatctttttcatgatgatattcaccatgtgcgcaaaagcatggaagtcaacttcctcagttacgtggtcctgactgtagctgccttgcccatgctgaagcagagcaatggaagcattgttgtcgtctcctctctggctgggaaagtggcttatccaatggttgctgcctattctgcaagcaagtttgctttggatgggttcttctcctccatcagaaaggaatattcagtgtccagggtcaatgtatcaatcactctctgtgttcttggcctcatagacacagaaacagccatgaaggcagtttctgggatagtccatatgcaagcagctccaaaggaggaatgtgccctggagatcatcaaagggggagctctgcgccaagaagaagtgtattatgacagctcactctggaccactcttctgatcagaaatccatgcaggaagatcctggaatttctctactcaacgagctataatatggacagattcataaacaagtaggaactccctgagggctgggcatgctgagggattttgggactgttctgtctcatgtttatctgagctcttatctatgaagacatcttcccagagtgtccccagagacatgcaagtcatgggtcacacctgacaaatggaaggagttcctctaacatttgcaaaatggaaatgtaataataatgaatgtcatgcaccgctgcagccagcagttgtaaaattgttagtaaacataggtataattaccagatagttatattaaatttatatcttatatataataatatgtgatgattaatacaatattaattataataaaggtcacataaactttataaattcataactggtagctataacttgagcttattcaggatggtttctttaaaaccataaactgtacaaatgaaatttttcaatatttgtttcttaaaaaaaaaaaaaaaaaa

SEQ ID NO: 4 - DNA, 1939 bp, Homo sapiens
ccctctcgcgccccaggccggtgtacccccgcactccgcgccccggcctagaagctctctctccccgctccccggcccggcccccgccccgccccgccccagcccgctgggccgccatggagcgctggccttggccgtcgggcggcgcctggctgctcgtggctgcccgcgcgctgctgcagctgctgcgctcagacctgcgtctgggccgcccgctgctggcggcgctggcgctgctggccgcgctcgactggctgtgccagcgcctgctgcccccgccggccgcactcgccgtgctggccgccgccggctggatcgcgttgtcccgcctggcgcgcccgcagcgcctgccggtggccactcgcgcggtgctcatcaccggctgtgactctggttttggcaaggagacggccaagaaactggactccatgggcttcacggtgctggccaccgtattggagttgaacagccccggtgccatcgagctgcgtacctgctgctcccctcgcctaaggctgctgcagatggacctgaccaaaccaggagacattagccgcgtgctagagttcaccaaggcccacaccaccagcaccggcctgtggggcctcgtcaacaacgcaggccacaatgaagtagttgctgatgcggagctgtctccagtggccactttccgtagctgcatggaggtgaatttctttggcgcgctcgagctgaccaagggcctcctgcccctgctgcgcagctcaaggggccgcatcgtgactgtggggagcccagcgggggacatgccatatccgtgcttgggggcctatggaacctccaaagcggccgtggcgctactcatggacacattcagctgtgaactccttccctggggggtcaaggtcagcatcatccagcctggctgcttcaagacagagtcagtgagaaacgtgggtcagtgggaaaagcgcaagcaattgctgctggccaacctgcctcaagagctgctgcaggcctacggcaaggactacatcgagcacttgcatgggcagttcctgcactcgctacgcctggccatgtccgacctcaccccagttgtagatgccatcacagatgcgctgctggcagctcggccccgccgccgctattaccccggccagggcctggggctcatgtacttcatccactactacctgcctgaaggcctgcggcgccgcttcctgcaggccttcttcatcagtcactgtctgcctcgagcactgcagcctggccagcctggcactaccccaccacaggacgcagcccaggacccaaacctgagccccggcccttccccagcagtggctcggtgagccatgtgcacctatggcccagccactgcagcacaggaggctccgtgagcccttggttcctccccgaaaacccccagcattacgatcccccaagtgtcctggaccctggcctaaagaatcccacccccacttcatgcccactgccgatgcccaatccaggcccggtgaggccaaggtttcccagtgagcctctgcgcctctccactgtttcatgagcccaaacaccctcctggcacaacgctctaccctgcagcttggagaactccgctggatggggagtctcatgcaagacttcactgcagcctttcacaggactctgcagatagtgcctctgcaaactaaggagtgactaggtgggttggggaccccctcaggattgtttctcggcaccagtgcctcagtgctgcaattgagggctaaatcccaagtgtctcttgactggctcaagaattagggccccaactacacacccccaagccacagggaagcatgtactgtacttcccaattgccacattttaaataaagacaaatttttatttcttctaaaaaaaaaaaaaaaaaaaaaaaaaaaaaaaaaaaaaaaaaaaaaaaaaaaaaaaaaaa

SEQ ID NO: 5 - DNA, 25 bp, Artificial
  Synthetic primer
gcatacgggtcctggcatcttgtcc

SEQ ID NO: 6 - DNA, 25 bp, Artificial
  Synthetic primer
atggtgatcttcttgctggtcttgc

SEQ ID NO: 7 - DNA, 20 bp, Artificial
  Synthetic primer
gacagcgaggtcaaaagaaa

SEQ ID NO: 8 - DNA, 21 bp, Artificial
  Synthetic primer
gtcctcccatgagctttcctg

SEQ ID NO: 9 - DNA, 21 bp, Artificial
  Synthetic primer
ccaccgtattggagttgaaca

SEQ ID NO: 10 - DNA, 19 bp, Artificial
  Synthetic primer
cgcggctaatgtctcctgg

SEQ ID NO: 11 - DNA, 21 bp, Artificial
  Synthetic primer
cacaggggtcttagtcgtgac

SEQ ID NO: 12 - DNA, 21 bp, Artificial
  Synthetic primer
ccaggtgaatgaacccacaca

SEQ ID NO: 13 - DNA, 19 bp, Artificial
  Synthetic primer
actggcgagcgtcatcttc

SEQ ID NO: 14 - DNA, 23 bp, Artificial
  Synthetic primer
tcctcgttataggtggagtcttg

SEQ ID NO: 15 - DNA, 21 bp, Artificial
  Synthetic primer
gcagagcacaaggatcagttc

SEQ ID NO: 16 - DNA, 21 bp, Artificial
  Synthetic primer
ggcagctactgttgatgttgc